(19) United States
(12) Patent Application Publication
(10) Pub. No.: US 2022/0213439 A1
(43) Pub. Date: Jul. 7, 2022

(54) SYSTEMS AND METHODS FOR MODULATING A CELL PHENOTYPE

(71) Applicant: XCELL BIOSCIENCES, INC., San Francisco, CA (US)

(72) Inventors: James LIM, San Francisco, CA (US); Quynh Anh Thuc LU, San Francisco, CA (US); Yunmin LI, San Francisco, CA (US); Brian FETH, San Francisco, CA (US)

(21) Appl. No.: 17/605,331

(22) PCT Filed: Apr. 30, 2020
(86) PCT No.: PCT/US2020/030706

§ 371 Date: Oct. 21, 2021

Related U.S. Application Data
(60) Provisional application No. 62/840,782, filed on Apr. 30, 2019.

(51) Int. Cl. C12N 5/0783; C12N 5/078; G01N 33/50; A61P 35/00; A61K 35/14; A61K 35/17; A61K 39/395

(52) U.S. Cl. CPC C12N 5/0638; C12N 5/0634; G01N 33/5011; C12N 2500/02; A61K 35/14; A61K 35/17; A61K 39/39558; A61P 35/00

(57) ABSTRACT

The present disclosure relates to a method of modulating a phenotype a source population of cells to assume a desired phenotype. The method includes culturing the source cell population within an incubator configured to regulate the variable atmospheric parameters of oxygen level and total atmospheric pressure level.

### CROSS-REFERENCE

This application claims priority to U.S. Provisional Application No. 62/840,782, filed on Apr. 30, 2019, which is incorporated herein by reference in its entirety.

### BACKGROUND

The use of cells, such as immune system cells, tumor cells, and stem cells, for research, diagnostic, drug screening, or therapeutic purposes is an area of interest, and accordingly, methods to isolate and expand cell populations well-suited for these purposes could be useful for various biological applications. In some instances, cells of a particular type can assume more than one phenotype by virtue of the state of development or differentiation, or by virtue of the influence of any number of environmental factors.

### SUMMARY OF THE INVENTION

In some embodiments, the invention provides a method of culturing a cell for enhanced cytotoxicity comprising culturing the cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure, wherein the cell is a peripheral blood mononuclear cell (PBMC), a pan T-cell, a regulatory T-cell (Treg), or a natural killer (NK) cell.

In some embodiments, the invention provides a method of treating a tumor in a subject in need thereof, the method comprising culturing a cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure and after the culturing, administering the cell to the subject wherein the cell is a peripheral blood mononuclear cell (PBMC), a pan T-cell, a regulatory T-cell (Treg), or a natural killer (NK) cell.

In some embodiments, the invention provides a method for determining efficacy of an anti-cancer agent, the method comprising: (a) culturing a cell that is selected from the group consisting of peripheral blood mononuclear cell (PBMC), a pan T-cell, a regulatory T-cell (Treg), or a natural killer (NK) cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure and after the culturing, (b) contacting a tumor cell with the cell and the anti-cancer agent; and (c) measuring cytotoxicity against the tumor cell after at least about five days, thereby determining the efficacy of the anti-cancer agent against the tumor cell.

In some embodiments, the invention provides a method of enriching a cell subpopulation from a source population of pan T-cells, the method comprising culturing the source population under 1% to about 15% oxygen and a pressure condition of no more than about 2 PSI above atmospheric pressure, wherein the cell subpopulation comprises CD8+ cells or CD4+ cells.

In some embodiments, the invention provides a method of treating a tumor in a subject in need thereof, the method comprising culturing a cell under about 1% to about 15% oxygen and a pressure condition of no more than about 2 PSI above atmospheric pressure at least until expression of IL-6 and IFN-γ is increased as compared to expression of the IL-6 and IFN-γ at a culturing condition of about 18% oxygen and 0 PSI above atmospheric pressure and after the culturing, administering the cell to the subject wherein the cell is a pan T-cell.

In some embodiments, the invention provides a method of modulating a phenotype of at least a subset of a source population of cells, the method including: culturing the source cell population in a liquid medium within a cell culture incubator that is configured to be able to regulate at least two variable atmospheric condition parameters within the incubator independently of a respective ambient atmospheric condition, wherein two of the variable atmospheric parameters are an oxygen level and a total atmospheric pressure level; regulating at least one of the oxygen level and the total atmospheric pressure level within the incubator such that at least one of the oxygen level or the total atmospheric pressure level differs from the respective ambient level; and as a consequence of the regulating of the variable atmospheric condition parameters, driving expression of a phenotypic parameter of the source population, over an incubation period, from a first phenotype toward a second phenotype, wherein the first phenotype of the subset cell population is that which would be expressed under an atmospheric condition in which the variable atmospheric condition parameters within the incubator were substantially the same as ambient atmospheric conditions, and wherein the second phenotype of the subset cell population is expressed as a consequence of exposure to the variable atmospheric conditions, as regulated by the incubator.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows expression of cytokines under standard culture conditions (STD), 15% O₂ at 2 PSI above atmospheric pressure (15% O₂+2 PSI), and at 15% O₂ at atmospheric pressure (15% O₂+0 PSI).

FIG. 1B shows a bar graph of data from the same study whose results are shown in FIG. 1A, the bar graph shows the expression of IFN-gamma, IL-6, IL-10, TGF-beta-1, and TNF-alpha under standard culture conditions (STD), 15% O₂ at 2 PSI above atmospheric pressure (15% O₂+2PSI), and at 15% O₂ at atmospheric pressure (15% O₂+0PSI).

FIG. 2 illustrates the effects of culturing under standard conditions (STD), or under 1% O₂ at 2 PSI above ambient pressure, on the tumor cell killing efficacy of PBMCs treated with Nivolumab (Nivo) or Pembrolizumab (Pembro).

FIG. 3A illustrates growth curves of primary T cells under varying conditions of oxygen and pressure. Conditions shown are standard conditions of 20% O₂ at atmospheric pressure, 15% O₂ at atmospheric pressure (15% O2+0PSI), 15% O₂ at 2 PSI above atmospheric pressure (15% O2+2PSI, 5% O₂ at atmospheric pressure (5% O2+0PSI), 5% O₂ at 2 PSI above atmospheric pressure (5% O2+2PSI, 1% O₂ at atmospheric pressure (1% O2+0PSI), and, 1% O₂ at 2 PSI above atmospheric pressure (1% O2+2PSI).

FIG. 3B illustrates the total doublings of Primary T cells at day 14, per the growth curves of FIG. 3A.

FIG. 3C summarizes the fold-expansion data of FIGS. 3A and 3B.

FIG. 4A shows representative flow cytometer plots of CD8 and CD4 expression in T cells under standard conditions (STD), 15% O₂ at atmospheric pressure (15% O2), 5% O₂ at atmospheric pressure (5% O2), 1% O₂ at atmospheric pressure (1% O2), 15% O₂ at 2 PSI above atmospheric pressure (15% O2+2PSI, 5% O₂ at 2 PSI above atmospheric pressure (5% O2+2PSI, and 1% O₂ at 2 PSI above atmospheric pressure (1% O2+2PSI).

FIG. 4B illustrates the relative presence of CD8+ cells at day 14 (per data of FIG. 4A) under varying conditions of oxygen and pressure.

FIG. 4C illustrates the relative presence of CD4+ cells at day 14 (per data of FIG. 4A) under varying conditions of oxygen and pressure.

FIG. 4D summarizes the relative presence of C8+ cells at day 14 (per data of FIG. 4B) under varying conditions of oxygen and pressure.

FIG. 4E summarizes the relative presence of C4+ cells at day 14 per data of FIG. 4B) under varying conditions of oxygen and pressure.

FIG. 5A illustrates the level of IL-10 expression in T-cells under varying conditions of oxygen and pressure relative to standard conditions.

FIG. 5B illustrates the level of IL-6 expression in T-cells under varying conditions of oxygen and pressure relative to standard conditions.

FIG. 6A illustrates the expression of granzyme B (GZMB) expression in T-cells under varying conditions of oxygen and pressure.

FIG. 6B illustrates the expression of perforin expression in T-cells under varying conditions of oxygen and pressure.

FIG. 7 illustrates the size (cell volume) of T-cells under varying conditions of oxygen and pressure.

FIG. 8A illustrates PD1 expression under varying conditions of oxygen and pressure.

FIG. 8B illustrates CTLA4 expression under varying conditions of oxygen and pressure.

FIG. 9A illustrates IFN-gamma expression under varying conditions of oxygen and pressure.

FIG. 9B illustrates IL-6 expression under varying conditions of oxygen and pressure.

FIG. 9C illustrates IL-10 expression under varying conditions of oxygen and pressure.

FIG. 10A illustrates the relative frequency of FOXP3+ Treg cells within the cell populations cultured under a hypoxic and hyperbaric condition vs. standard culture condition.

FIG. 10B illustrates a flow cytometer analysis of the relative distribution of FOXP3+ Treg cells within populations cultured under a hypoxic and hyperbaric condition vs. a standard culture condition.

FIG. 10C illustrates a flow cytometry analysis of side scatter area (SSC-A) vs. FOXP3+ of Treg cells cultured under a standard culture condition.

FIG. 10D illustrates a flow cytometry analysis of side scatter area (SSC-A) vs. FOXP3+ of Treg cells cultured under hyperbaric and hypoxic conditions (5% O₂ at 2 PSI above atmospheric pressure).

FIG. 10E illustrates the relative percentages of FOXP3+ cells in hypoxic and hyperbaric condition vs. a standard culture condition.

FIG. 11 illustrates an X-Y graphic representation of an oxygen level parameter (X-axis) and a pressure level parameter (Y-axis) for gaseous conditions in a cell culture incubator, showing two particular oxygen-pressure (O-P) conditions: O-P Condition 1 and O-P Condition 2.

FIG. 12 illustrates an X-Y-Z graphic representation of an oxygen level parameter (X-axis), a pressure level parameter (Y-axis), and time (Z-axis) for gaseous conditions in a cell culture incubator, with O-P Condition 1 shown at Time Point 1 and O-P Condition 2 shown at Time Point 2, having changed during the duration from Time Point 1 to Time Point 2.

FIG. 13A shows a biphasic frequency distribution profile of cells with respect to a measurement of a cell culture parameter, wherein movement or reformation of the population from one peak to another is facilitated or driven by atmospheric conditions.

FIG. 13B shows a frequency distribution profile of cells with respect to a measurement of a cell culture parameter, wherein a cell population becomes more homogenous as a consequence of regulating atmospheric conditions.

FIG. 13C shows a frequency distribution profile of cells with respect to a measurement of a cell culture parameter, wherein the population has an apparent tendency to drift from a central median one direction and/or another, and wherein such phenotypic drift is prevented by an optimal regulation of atmospheric conditions.

FIG. 13D shows a two-factor (oxygen level and pressure level) experiment designed to determine optimal atmospheric conditions that support a targeted or desired phenotype.

FIG. 13E shows an analysis of a multi-factor experiment (oxygen level, pressure level, bioactive agent) that is designed to optimize atmospheric conditions that optimize phenotypic expression of a cell population for an ability to measure the effect of a bioactive agent.

FIG. 13F illustrates a schematic representation of a two-phase manufacturing process optimized for maximal product yield.

FIG. 14 illustrates a method flow diagram showing a method of modulating the phenotype of a source population of cells.

FIG. 15 illustrates a method flow diagram showing a method of increasing phenotypic homogeneity within a cell population.

FIG. 16 illustrates a method flow diagram showing a method of stabilizing a phenotype of population of cells.

FIG. 17 illustrates a method flow diagram showing a method of determining atmospheric parameter values that favor expression of a desired phenotype.

FIG. 18 illustrates a method flow diagram showing a method of optimizing an immune cell-based cell culture assay for evaluating an immune cell-directed bioactive agent.

FIG. 19 illustrates a method flow diagram showing a method of modulating phenotypic expression of a cell culture population to achieve a targeted phenotype in a manufacturing context.

FIG. 20 illustrates a method flow diagram showing a method of modulating phenotypic expression of a cell culture population to achieve an optimal manufacturing process efficiency.

FIG. 21 illustrates a method flow diagram showing a product being made by way of modulating a phenotype of a cell population.

FIG. 22 illustrates a method flow diagram showing a method of testing the efficacy of an anti-cancer drug on patient-derived cancer cells in vitro.

FIG. 23 shows a process for isolating and analyzing cells based on a method described herein.

### DETAILED DESCRIPTION OF THE INVENTION

The method disclosed herein can allow for, for example, conducting of experiments, drug screening, and treatment of patients to be performed at conditions that simulate in vivo conditions. Early-stage tests during drug discovery can be performed on cell lines that can be maintained and expanded over a period of time, in some cases many years, under conditions that rarely reflect in vivo settings. Conventional cell culture incubators housing cell lines are not often capable of fully reproducing the native conditions of the cells, and in some cases, even small environmental changes can alter the expression of genes and proteins in the cells. In some cases, these differences in gene and protein expression signatures that are induced by cell culture conditions can contribute to changes in drug sensitivity.

Described herein are methods for culturing cells under physiological conditions. Such methods can aid drug discovery by allowing the generation of more physiologically relevant data than is possible using conventional techniques and systems. In some cases, data produced by such methods can be used to identify and advance the best drug candidate(s) to clinical trial. A method described herein can allow for culturing cells under customized conditions, which can then be introduced into a patient to, for example, increase efficacy of co-administered drug or increase cytotoxicity of the cell against a tumor cell in the patient.

The methods provided herein can comprise culturing cells under physiological oxygen and pressure conditions. Important biological traits such as hypoxia and pressure can be essential in mirroring physiological conditions. These conditions can be customized based on cell type or utility. In some embodiments, such approaches can more closely mimic the native microenvironment of cells, and can allow them to function as the cells would function in vivo. In some embodiments, testing compounds on cells cultured using methods provided herein can allow discovery and development processes to become more efficient and effective.

For example, immune cells, such as T cells, can be cultured using methods provided herein. For example, activity of such T cells can outperform that of T cells cultured using conventional methods, suggesting that only a small fraction of patient derived T cells may work to kill cancer cells when injected into a patient. For example, T cell activity can be diminished during bioproduction in traditional incubators, rendering the T-cells less effective. By culturing such cells using the methods provided herein, the methods can maintain optimal fitness of the T cells, which can make the cells more effective at killing cancer cells when reintroduced to patients.

FIG. 23 depicts a method disclosed herein. FIG. 23 shows a process 100 for capturing and enriching target cell subpopulations, in accordance with an embodiment disclosed herein. The process 100 can comprise obtaining a sample 110 from a subject 105; separating one or more components of the sample 115 to obtain a heterogeneous cell population; contacting the heterogeneous cell population with a substrate 120 (e.g., plating the heterogeneous cell population onto the substrate); incubating the heterogeneous cell population under conditions sufficient to allow growth of a target cell subpopulation, wherein the conditions sufficient to allow growth of a target cell subpopulation can comprise incubating with enrichment media 125 in an apparatus 130; incubating the samples for a time 135, wherein the time 135 is sufficient for cell division to occur. The cells can be monitored using an imaging system 140 (e.g. a microscope). Data from monitoring with an imaging system can be processed using analysis software 145. Analysis software 145 can create an output of results 150. The cells and/or results 150 can be used for diagnosis, prognosis, or the monitoring of a disease condition 155, to direct a treatment regimen for a subject 160, and/or for research or any other suitable purpose, such as therapy, diagnosis, or treatment regimen determination 165.

Gaseous or atmospheric conditions and dissolved gas levels in liquid medium can be important in controlling changes in the phenotype of cultured cells. Various atmospheric conditions can drive phenotypic changes or stabilize a particular phenotype.

With regard to stem cells and the therapeutic potential of stem cells, progression of a cell lineage potency status can be influenced from, for example, the pluripotent or nearly pluripotent status of a stem cell toward an intermediately differentiated or fully differentiated state, or alternatively, to drive a cell lineage from a differentiated state toward a pluripotent state. In vitro conditions, such as the presence of any of a multitude of reprogramming factors, induction factors, growth factors, and cytokines can promote movement of a cell lineage phenotype in the directions of either increasing differentiation or (oppositely) increasing potency-level.

A cell phenotype that can be affected by a method disclosed herein can include, for example, cell morphology, dimension, adherent properties, electrical properties, metabolic activity, or migratory behavior. In some cases, phenotypic differences between differentiated cell states can be associated with a difference in potency level, which can manifest as difference in messenger RNA expression of the cellular genome, or rates of protein transcription from expressed RNA.

Physical factors, such as availability of substrates or 3D scaffolding arrangements, can have important influences on cellular phenotypic expression. In some embodiments, such physical factors can manifest as differences in morphology or function.

Additionally, the environment of cells in the body can be different than conditions within a conventional cell culture incubator, wherein the oxygen level and the total gas pressure levels are substantially similar to ambient conditions (e.g., conditions outside the body). In contrast, local anatomical compartments or microenvironments, (e.g., a tumor microenvironment) in the body can be hypoxic (oxygen level is less than the ambient level) or hyperbaric (total atmospheric pressure is greater than the ambient level). Hypoxia can be an influencing atmospheric factor with a host of effects on particular types of cells, as mediated by hypoxia-inducible factors. The effects of total atmospheric pressure on cells are in some cases less well understood than the effects of hypoxia, at least in part because it is relatively easy to create different levels of oxygen in an in vitro environment, but there are few available incubator options that can controllably vary the internal atmospheric pressure.

### Methods of Culturing Cells.

Provided herein are methods for culturing cells. Cells can be cultured (e.g., in vitro) at an oxygen level and total gas pressure that can be reflective of the conditions of a similar cell in a body (e.g., in vivo). In some embodiments, an oxygen level or total gas pressure can be that of a specific tissue or organ, that of a disease state, or that of a healthy state. In some embodiments, an oxygen level or total gas pressure that cells can be cultured in can be of blood, a tumor, or another compartment, tissue, or organ in a body.

Methods of culturing cells can comprise incubating a source cell population in a cell culture incubator that can be configured to operate an atmospheric condition-controlling incubator program. In some embodiments, the program can control atmospheric conditions that can optimize expression of a targeted phenotype. Such a program can include set point ranges for (1) an oxygen level and (2) a total gas pressure level and can regulate oxygen level and total gas pressure within the incubator in accordance with the atmospheric condition set point ranges. The cell population can be cultured in accordance with said atmospheric condition set point ranges to yield an expanded population of cells that express a desired phenotype. In some embodiments, the cultured cells can have therapeutic or cytotoxic properties.

Also provided herein are methods for culturing peripheral blood mononuclear cells (PBMC), for example for enhanced cytotoxicity. In some embodiments, the methods can comprise culturing a PMBC isolated from a donor organ at least until expression of a cytokine is altered relative to expression of the cytokine at a control culturing condition of the PBMC.

A PBMC cell can be of a source population of immune cells, such as a PBMC population from a subject (e.g., a human, a patient, or an animal subject). A PBMC can be a peripheral blood cell having a round nucleus. In some embodiments, a PBMC can be a lymphocyte, (e.g., a T cell, a B cell, or a NK cell), a monocyte, or a dendritic cell. In some embodiments, after culturing, a PBMC can show an increased or decreased cytokine expression. In some embodiments, after culturing a PBMC can show an increased ability to detect, bind, or kill a target cell (e.g., a cancer cell). In some embodiments, such increased ability to detect, bind, or kill a target cell can be enhanced by or observed in conjugation with, a therapeutic.

In some embodiments, the oxygen level during culturing can be regulated to a hypoxic level with respect to the ambient oxygen level. In some embodiments, the total atmospheric pressure can be regulated to a hyperbaric level with respect to the ambient atmospheric pressure. In some embodiments of the method, the oxygen level can be regulated to a hypoxic level with respect to the ambient oxygen level, and the total atmospheric pressure is regulated to a hyperbaric level with respect to the ambient atmospheric pressure. In embodiments, the atmospheric pressure and the oxygen level are regulated independently of each other such that a hypoxic oxygen level prevails in spite of an overall hyperbaric condition.

In some embodiments, the PBMC can be cultured at an oxygen level of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or a range between any two foregoing values. In some embodiments, the PMBC can be cultured at about 1% to about 15% oxygen.

In some embodiments, a PBMC can be cultured at a pressure condition of about 0 pounds per square inch (PSI) above atmospheric pressure, about 0.5 PSI above atmospheric pressure, about 1 PSI above atmospheric pressure, about 1.5 PSI above atmospheric pressure, about 2 PSI above atmospheric pressure, about 2.5 PSI above atmospheric pressure, or about 3 PSI above atmospheric pressure, or a range between any two foregoing values. In some embodiments, a PMBC can be cultured at a pressure condition of no more than about 0.5 PSI above atmospheric pressure, no more than about 1 PSI above atmospheric pressure, no more than about 1.5 PSI above atmospheric pressure, no more than about 2 PSI above atmospheric pressure, no more than about 2.5 PSI above atmospheric pressure, or no more than about 3 PSI above atmospheric pressure. In some embodiments, a PMBC can be cultured at a pressure condition of at least about 0.5 PSI above atmospheric pressure, at least about 1 PSI above atmospheric pressure, at least about 1.5 PSI above atmospheric pressure, at least about 2 PSI above atmospheric pressure, at least about 2.5 PSI above atmospheric pressure, or at least about 3 PSI above atmospheric pressure. In some embodiments, for example, a PBMC can be cultured at a pressure condition of no more than about 2 PSI above atmospheric pressure. In some embodiments, a PBMC can be cultured at a pressure condition of at least about 1 PSI above atmospheric pressure.

In some embodiments, a PBMC can be cultured at a given oxygen level and a given pressure condition, such as an oxygen level and pressure condition provided above. In some embodiments, for example, the PBMC can be cultured at about 1% to about 15% oxygen, and the pressure condition can be no more than about 2 PSI above atmospheric pressure. In some embodiments, the PBMC can be cultured at about 15% oxygen and a pressure condition of no more than 2 PSI above atmospheric pressure. In some embodiments, the PBMC can be cultured at from about 1% to about 15% oxygen and a pressure condition of at least about 1 PSI above atmospheric pressure. In some embodiments, the PBMC can be cultured at about 15% oxygen and a pressure condition of about 1 PSI above atmospheric pressure.

Control culturing conditions can comprise standard culturing conditions. In some embodiments, control culturing conditions can comprise an ambient (e.g., of the room or atmospheric) oxygen level or pressure condition.

A control culturing condition can comprise an oxygen level of about 15%, about 16%, about 17%, about 18%, about 19%, or about 20%, or a range between any two foregoing values. In some embodiments, a control culturing condition can comprise an oxygen level of at least about 15%, at least about 16%, at least about 17%, at least about 18%, at least about 19%, or at least about 20%.

A control culturing condition can comprise a pressure condition of about 0 PSI above atmospheric pressure, about 0.5 PSI above atmospheric pressure, about 1 PSI above atmospheric pressure, or a range between any two foregoing values. In some embodiments, for example, a control culturing condition can comprise a pressure condition of about 0 PSI above atmospheric pressure. In some embodiments, a control culturing condition can comprise a pressure condition of no more than 0.5 PSI above atmospheric pressure or no more than 1 PSI above atmospheric pressure.

In some embodiments, a control culturing condition can comprise a combination of a given oxygen level and a given pressure condition, such as an oxygen level and pressure condition of a control culturing condition provided above. For example, in some embodiments, control culturing condition can comprise about 18% oxygen and a pressure condition of 0 PSI above atmospheric pressure.

A PBMC can be cultured at least until expression of a cytokine in the PBMC is altered relative to expression of the cytokine at a control culturing condition of the PBMC. A cytokine can be a small protein that can play a role in cell signaling. In some embodiments, a cytokine can be involved in autocrine, paracrine, or endocrine signaling pathways. In some embodiments, a cytokine can be an immunomodulating agent. Cytokines can include chemokines, interferons, interleukins, lymphokines, or tumor necrosis factors. Examples of cytokines can include, for example, IL-2, IL-4, IL-6, IL-7, IL-10, IL-12, IL-15, IL-17, IL-21, CD40 L (CD154), lymphotoxin (LT, TNFβ), interferon-α, interferon-β, interferon-γ, C-CSF, GM-CSF, or M-CSF. In some embodiments, other cytokines can have altered expression. In some embodiments, the cytokine can be a marker of a non-differentiated cell. In some embodiments, the cytokine can be a marker of a differentiated cell.

Alteration of expression of a cytokine can comprise an increase or decrease in expression. In some embodiments, expression of a cytokine can be increased by at least about 5%, at least about 10%, at least about 20%, at least about 30%, at least about 40%, at least about 50%, at least about 100%, at least about 200%, at least about 300%, at least about 400%, at least about 500%, or at least about 1000% in the cultured PBMC compared with expression of a same cytokine in a cell cultured at a control culturing condition. In some embodiments, expression of a cytokine can be decreased by at least about 5%, at least about 10%, at least about 20%, at least about 30%, at least about 40%, at least about 50%, at least about 100%, at least about 200%, at least about 300%, at least about 400%, at least about 500%, or at least about 1000% in the cultured PBMC compared with expression of a same cytokine in a cell cultured at a control culturing condition.

In some embodiments, altered cytokine expression can comprise altered gene expression, such as an increase or decrease in expression of mRNA that codes for a cytokine. Altered expression of mRNA can for example comprise increased or decreased transcription of DNA to mRNA. In some cases, altered expression of mRNA can comprise increased or decreased degradation of mRNA coding for a cytokine.

Gene expression can be determined by any acceptable method. For example, gene expression can be determined using ISH, FISH, northern blot, PCR, RT-PCR, q-PCR, p-RT-PCR, or another method. RNA expression can be determined as an absolute expression (e.g., number of mRNA transcripts per cell) or a relative expression (e.g., number of mRNA transcripts compared with the number of mRNA transcripts of a housekeeping gene in the same cell, or a number of mRNA transcripts compared with the number of same mRNA transcripts of a cell cultured under control conditions).

In some embodiments, altered cytokine expression can comprise altered protein expression, such as an increase or decrease in cytokine produced or detected. Altered protein expression can for example comprise increased or decreased translation of mRNA to protein. In some cases, altered protein expression can comprise increased or decreased degradation or inactivation of the protein.

Protein expression can be determined by any acceptable method. For example, protein expression can be determined by immunoassay (e.g., ELISA), western blot, dot blot, chromatography, spectrophotometry, or another method. Protein expression can be determined as an absolute expression (e.g., number of protein molecules per cell) or a relative expression (e.g., number of protein molecules compared with the number of protein molecules of a housekeeping protein in the same cell, or number of protein molecules compared with the number of same protein molecules of a cell cultured under control conditions).

In some embodiments, the cytokine can be IL-10. In some such embodiments, the expression of IL-10 can be decreased. In some embodiments, the cytokine can be TNF-α. In some such embodiments, the expression of TNF-α can be increased. In some embodiments, the cytokine can be IL-6. In some such embodiments, the expression of IL-6 can be decreased. In some embodiments, the cytokine can comprise IFN-γ. In some such embodiments, the expression of IFN-γ can be decreased. In some embodiments, the cytokine can be TGF-β1, and the expression of TGF-β1 can be increased.

In some embodiments, the cytotoxicity of the PBMC can be increased, for example compared with a PBMC cultured under control conditions such as control conditions provided above. In some embodiments, cytotoxicity of the PBMC can comprise the ability of the PBMC to recognize, bind, neutralize, or kill a target cell. Cytotoxicity of a PBMC can refer to the ability of the PBMC to recognize, bind, neutralize, or kill a tumor cell, a metastatic cell, a cancer cell, a bacterial cell, or another type of cell. For example, a PBMC can display cytotoxicity against a carcinoma cell, a sarcoma cell, a melanoma cell, a lymphoma cell, or a leukemia cell.

In some embodiments, the cytotoxicity of the PBMC can be increased by at least about 10%, at least about 15%, at least about 20%, at least about 25%, at least about 30%, at least about 35%, at least about 40%, at least about 45%, at least about 50%, or a range between any two foregoing values, relative to the control culturing condition of the PBMC. For example, in some embodiments, the cytotoxicity of the PBMC can be increased by about 20% relative to the control culturing condition of the PBMC.

Cytotoxicity of a PBMC can be determined using a cytotoxicity assay. In some embodiments, a cytotoxicity assay can be performed in vitro. A cytotoxicity assay can comprise exposing a cell or a plurality of cells to a PBMC cultured as described herein and detecting whether the cell(s) is alive or dead after the exposure. In some embodiments, detection of dead cells can be accomplished by measuring movement of molecules either into or out of cells across membranes. In some embodiments, membranes of dead cells can be leaky and cannot be repaired. For example, detection of cytoplasmic markers in the culture medium surrounding the cell(s) can indicate a loss of membrane integrity and thus a dead cell. Such a marker can be a naturally existing marker, such as an enzyme, or can be introduced artificially, such as a radioactive or fluorescent marker loaded into cells prior to exposure to PBMCs.

In some embodiments, cytotoxicity can be determined in vivo. For example, cytotoxicity can be determined by injection of a PBMC into a subject having a tumor, such as a human subject or an animal subject (e.g., a mouse, hamster, rat, guinea pig, monkey, cat, dog, rabbit, or other animal). In some such cases, cytotoxicity can be determined by measuring reduction in tumor mass, reduction in tumor cells, or reduction in metastatic cells.

### Methods for Treating a Tumor.

Also provided herein are methods for treating a tumor in a subject. Methods can comprise culturing a PBMC, or any other cell disclosed herein, under conditions described above. For example, a method can comprise culturing an isolated PBMC under an oxygen level of about 1% to about 15%, and a pressure condition of no more than about 2 PSI at least until expression of a cytokine is altered relative to the expression of the cytokine at a control culturing condition of the PBMC. In some embodiments, the method can further comprise administering the PBMC to the subject.

In some embodiments, the PBMC can be cultured at an oxygen level of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or a range between any two foregoing values. In some embodiments, the PMBC can be cultured at about 1% to about 15% oxygen.

In some embodiments, a PBMC can be cultured at a pressure condition of about 0 pounds per square inch (PSI) above atmospheric pressure, about 0.5 PSI above atmospheric pressure, about 1 PSI above atmospheric pressure, about 1.5 PSI above atmospheric pressure, about 2 PSI above atmospheric pressure, about 2.5 PSI above atmospheric pressure, or about 3 PSI above atmospheric pressure, or a range between any two foregoing values. In some embodiments, a PMBC can be cultured at a pressure condition of no more than about 0.5 PSI above atmospheric pressure, no more than about 1 PSI above atmospheric pressure, no more than about 1.5 PSI above atmospheric pressure, no more than about 2 PSI above atmospheric pressure, no more than about 2.5 PSI above atmospheric pressure, or no more than about 3 PSI above atmospheric pressure. In some embodiments, a PMBC can be cultured at a pressure condition of at least about 0.5 PSI above atmospheric pressure, at least about 1 PSI above atmospheric pressure, at least about 1.5 PSI above atmospheric pressure, at least about 2 PSI above atmospheric pressure, at least about 2.5 PSI above atmospheric pressure, or at least about 3 PSI above atmospheric pressure. In some embodiments, for example, a PBMC can be cultured at a pressure condition of no more than about 2 PSI above atmospheric pressure. In some embodiments, a PBMC can be cultured at a pressure condition of at least about 1 PSI above atmospheric pressure.

In some embodiments, a PBMC can be cultured at a given oxygen level and a given pressure condition, such as an oxygen level and pressure condition provided above. In some embodiments, for example, the PBMC can be cultured at about 1% to about 15% oxygen, and the pressure condition can be no more than about 2 PSI above atmospheric pressure. In some embodiments, the PBMC can be cultured at about 15% oxygen and a pressure condition of no more than 2 PSI above atmospheric pressure. In some embodiments, the PBMC can be cultured at from about 1% to about 15% oxygen and a pressure condition of at least about 1 PSI above atmospheric pressure. In some embodiments, the PBMC can be cultured at about 15% oxygen and a pressure condition of about 1 PSI above atmospheric pressure.

Control culturing conditions can comprise standard culturing conditions. In some embodiments, control culturing conditions can comprise an ambient (e.g., of the room or atmospheric) oxygen level or pressure condition.

A control culturing condition can comprise an oxygen level of about 15%, about 16%, about 17%, about 18%, about 19%, or about 20%, or a range between any two foregoing values. In some embodiments, a control culturing condition can comprise an oxygen level of at least about 15%, at least about 16%, at least about 17%, at least about 18%, at least about 19%, or at least about 20%.

A control culturing condition can comprise a pressure condition of about 0 PSI above atmospheric pressure, about 0.5 PSI above atmospheric pressure, about 1 PSI above atmospheric pressure, or a range between any two foregoing values. In some embodiments, for example, a control culturing condition can comprise a pressure condition of about 0 PSI above atmospheric pressure. In some embodiments, a control culturing condition can comprise a pressure condition of no more than 0.5 PSI above atmospheric pressure or no more than 1 PSI above atmospheric pressure.

In some embodiments, a control culturing condition can comprise a combination of a given oxygen level and a given pressure condition, such as an oxygen level and pressure condition of a control culturing condition provided above. For example, in some embodiments, control culturing condition can comprise about 18% oxygen and a pressure condition of 0 PSI above atmospheric pressure.

A PBMC can be cultured at least until expression of a cytokine in the PBMC is altered relative to expression of the cytokine at a control culturing condition of the PBMC. A cytokine can be a small protein that can play a role in cell signaling. In some embodiments, a cytokine can be involved in autocrine, paracrine, or endocrine signaling pathways. In some embodiments, a cytokine can be an immunomodulating agent. Cytokines can include chemokines, interferons, interleukins, lymphokines, or tumor necrosis factors. Examples of cytokines can include, for example, IL-2, IL-4, IL-6, IL-7, IL-10, IL-12, IL-15, IL-17, IL-21, CD40 L (CD154), lymphotoxin (LT, TNFβ), interferon-α, interferon-β, interferon-γ, C-CSF, GM-CSF, or M-CSF. In some embodiments, other cytokines can have altered expression. In some embodiments, the cytokine can be a marker of a non-differentiated cell. In some embodiments, the cytokine can be a marker of a differentiated cell. In some embodiments, the altered cytokine can contribute to increased cytotoxicity of the PBMC.

Alteration of expression of a cytokine can comprise an increase or decrease in expression. In some embodiments, expression of a cytokine can be increased by at least about 5%, at least about 10%, at least about 20%, at least about 30%, at least about 40%, at least about 50%, at least about 100%, at least about 200%, at least about 300%, at least about 400%, at least about 500%, or at least about 1000% in the cultured PBMC compared with expression of a same cytokine in a cell cultured at a control culturing condition. In some embodiments, expression of a cytokine can be decreased by at least about 5%, at least about 10%, at least about 20%, at least about 30%, at least about 40%, at least about 50%, at least about 100%, at least about 200%, at least about 300%, at least about 400%, at least about 500%, or at least about 1000% in the cultured PBMC compared with expression of a same cytokine in a cell cultured at a control culturing condition.

In some embodiments, altered cytokine expression can comprise altered gene expression, such as an increase or decrease in expression of mRNA that codes for a cytokine. Altered expression of mRNA can for example comprise increased or decreased transcription of DNA to mRNA. In some cases, altered expression of mRNA can comprise increased or decreased degradation of mRNA coding for a cytokine.

Gene expression can be determined by any acceptable method. For example, gene expression can be determined using ISH, FISH, northern blot, PCR, RT-PCR, q-PCR, p-RT-PCR, or another method. RNA expression can be determined as an absolute expression (e.g., number of mRNA transcripts per cell) or a relative expression (e.g., number of mRNA transcripts compared with the number of mRNA transcripts of a housekeeping gene in the same cell, or a number of mRNA transcripts compared with the number of same mRNA transcripts of a cell cultured under control conditions).

In some embodiments, altered cytokine expression can comprise altered protein expression, such as an increase or decrease in cytokine produced or detected. Altered protein expression can for example comprise increased or decreased translation of mRNA to protein. In some cases, altered protein expression can comprise increased or decreased degradation or inactivation of the protein.

Protein expression can be determined by any acceptable method. For example, protein expression can be determined by immunoassay (e.g., ELISA), western blot, dot blot, chromatography, spectrophotometry, or another method. Protein expression can be determined as an absolute expression (e.g., number of protein molecules per cell) or a relative expression (e.g., number of protein molecules compared with the number of protein molecules of a housekeeping protein in the same cell, or number of protein molecules compared with the number of same protein molecules of a cell cultured under control conditions).

In some embodiments, the cytokine can be IL-10. In some such embodiments, the expression of IL-10 can be decreased. In some embodiments, the cytokine can be TNF-α. In some such embodiments, the expression of TNF-α can be increased. In some embodiments, the cytokine can be IL-6. In some such embodiments, the expression of IL-6 can be decreased. In some embodiments, the cytokine can comprise IFN-γ. In some such embodiments, the expression of IFN-γ can be decreased. In some embodiments, the cytokine can be TGF-β1, and the expression of TGF-β1 can be increased.

The subject can be a subject in need of treatment of a tumor, such as a subject having a tumor. In some embodiments, the subject can be a human, a mouse, a hamster, a guinea pig, a rat, a rabbit, a cat, or a dog. In some embodiments, the subject can have a single tumor. In some embodiments, the subject can also have metastases.

Administering can comprise providing one or more compositions (e.g., a composition comprising PBMCs, or a PBMC composition) to a subject in a manner that results in the composition being inside the patients body. Administration can be by any route, including, without limitation, locally, regionally, or systemically. Administration can be subcutaneous, intradermal, intravenous, intra-arterial, intraperitoneal, or intramuscular.

Some embodiments can comprise the use of the PBMCs described herein to manufacture a medicament for treating a condition, disease or disorder described herein. Medicaments can be formulated based on the physical characteristics of the subject needing treatment, and can be formulated in single or multiple formulations based on the stage of the tumor. Medicaments can be packaged in a suitable package with appropriate labels for the distribution to hospitals and clinics wherein the label is for the indication of treating a subject having a disease described herein. Medicaments can be packaged as a single or multiple units. Instructions for the dosage and administration of the compositions can be included with the packages as described below.

In some embodiments, the PBMC can be co-administered to the subject with an anti-cancer agent. In some embodiments, the anti-cancer agent can comprise a drug that can be clinically effective in reducing tumor burden, preventing or eliminating metastases, killing tumor cells, or preventing growth of tumor cells. In some embodiments, an anti-cancer agent can be a PD1 inhibitor. Examples of a PD1 inhibitor can include, for example, pembrolizumab or nivolumab.

In some embodiments, the anti-cancer agent can be co-administered by the same route as the PBMC composition. Administration can be by any route, including, without limitation, locally, regionally, or systemically. Administration can be subcutaneous, intradermal, intravenous, intra-arterial, intraperitoneal, or intramuscular.

In some embodiments, the anti-cancer agent can be co-administered at the same time as the PBMC composition. In some such embodiments, the anti-cancer agent can be included in the PBMC composition.

The anti-cancer agent can be co-administered before administration of the PBMC composition. In some embodiments, the anti-cancer agent can be administered immediately prior to administration of the PBMC composition. In some embodiments, the anti-cancer agent can be administered about 1 minute before the PBMC composition, about 5 minutes before the PBMC composition, about 15 minutes before the PBMC composition, about 30 minutes before the PBMC composition, about 1 hour before the PBMC composition, about 6 hours, before the PBMC composition, about 12 hours before the PBMC composition, or a range between any two foregoing values.

In some embodiments, the anti-cancer agent can be co-administered after administration of the PBMC composition. In some embodiments, the anti-cancer agent can be administered immediately after administration of the PBMC composition. In some embodiments, the anti-cancer agent can be administered about 1 minute after the PBMC composition, about 5 minutes after the PBMC composition, about 15 minutes after the PBMC composition, about 30 minutes after the PBMC composition, about 1 hour after the PBMC composition, about 6 hours after the PBMC composition, about 12 hours after the PBMC composition, or a range between any two foregoing values.

### Methods for Determining Efficacy of an Anti-Cancer Agent.

Also provided herein are methods for determining efficacy of an anti-cancer agent. In some embodiments, a method for determining the efficacy of an anti-cancer agent can comprise culturing a peripheral blood mononuclear cell until expression of a cytokine is altered relative to expression of the cytokine at a control culturing condition of the PBMC.

In some embodiments, the oxygen level during culturing can be regulated to a hypoxic level with respect to the ambient oxygen level. In some embodiments, the total atmospheric pressure can be regulated to a hyperbaric level with respect to the ambient atmospheric pressure. In some embodiments of the method, the oxygen level can be regulated to a hypoxic level with respect to the ambient oxygen level, and the total atmospheric pressure is regulated to a hyperbaric level with respect to the ambient atmospheric pressure. In embodiments, the atmospheric pressure and the oxygen level are regulated independently of each other such that a hypoxic oxygen level prevails in spite of an overall hyperbaric condition.

In some embodiments, the PBMC can be cultured at an oxygen level of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or a range between any two foregoing values. In some embodiments, the PMBC can be cultured at about 1% to about 15% oxygen.

In some embodiments, a PBMC can be cultured at a pressure condition of about 0 pounds per square inch (PSI) above atmospheric pressure, about 0.5 PSI above atmospheric pressure, about 1 PSI above atmospheric pressure, about 1.5 PSI above atmospheric pressure, about 2 PSI above atmospheric pressure, about 2.5 PSI above atmospheric pressure, or about 3 PSI above atmospheric pressure, or a range between any two foregoing values. In some embodiments, a PMBC can be cultured at a pressure condition of no more than about 0.5 PSI above atmospheric pressure, no more than about 1 PSI above atmospheric pressure, no more than about 1.5 PSI above atmospheric pressure, no more than about 2 PSI above atmospheric pressure, no more than about 2.5 PSI above atmospheric pressure, or no more than about 3 PSI above atmospheric pressure. In some embodiments, a PMBC can be cultured at a pressure condition of at least about 0.5 PSI above atmospheric pressure, at least about 1 PSI above atmospheric pressure, at least about 1.5 PSI above atmospheric pressure, at least about 2 PSI above atmospheric pressure, at least about 2.5 PSI above atmospheric pressure, or at least about 3 PSI above atmospheric pressure. In some embodiments, for example, a PBMC can be cultured at a pressure condition of no more than about 2 PSI above atmospheric pressure. In some embodiments, a PBMC can be cultured at a pressure condition of at least about 1 PSI above atmospheric pressure.

In some embodiments, a PBMC can be cultured at a given oxygen level and a given pressure condition, such as an oxygen level and pressure condition provided above. In some embodiments, for example, the PBMC can be cultured at about 1% to about 15% oxygen, and the pressure condition can be no more than about 2 PSI above atmospheric pressure. In some embodiments, the PBMC can be cultured at about 15% oxygen and a pressure condition of no more than 2 PSI above atmospheric pressure. In some embodiments, the PBMC can be cultured at from about 1% to about 15% oxygen and a pressure condition of at least about 1 PSI above atmospheric pressure. In some embodiments, the PBMC can be cultured at about 15% oxygen and a pressure condition of about 1 PSI above atmospheric pressure.

Control culturing conditions can comprise standard culturing conditions. In some embodiments, control culturing conditions can comprise an ambient (e.g., of the room or atmospheric) oxygen level or pressure condition.

A control culturing condition can comprise an oxygen level of about 15%, about 16%, about 17%, about 18%, about 19%, or about 20%, or a range between any two foregoing values. In some embodiments, a control culturing condition can comprise an oxygen level of at least about 15%, at least about 16%, at least about 17%, at least about 18%, at least about 19%, or at least about 20%.

A control culturing condition can comprise a pressure condition of about 0 PSI above atmospheric pressure, about 0.5 PSI above atmospheric pressure, about 1 PSI above atmospheric pressure, or a range between any two foregoing values. In some embodiments, for example, a control culturing condition can comprise a pressure condition of about 0 PSI above atmospheric pressure. In some embodiments, a control culturing condition can comprise a pressure condition of no more than 0.5 PSI above atmospheric pressure or no more than 1 PSI above atmospheric pressure.

In some embodiments, a control culturing condition can comprise a combination of a given oxygen level and a given pressure condition, such as an oxygen level and pressure condition of a control culturing condition provided above. For example, in some embodiments, control culturing condition can comprise about 18% oxygen and a pressure condition of 0 PSI above atmospheric pressure.

A PBMC can be cultured at least until expression of a cytokine in the PBMC is altered relative to expression of the cytokine at a control culturing condition of the PBMC. A cytokine can be a small protein that can play a role in cell signaling. In some embodiments, a cytokine can be involved in autocrine, paracrine, or endocrine signaling pathways. In some embodiments, a cytokine can be an immunomodulating agent. Cytokines can include chemokines, interferons, interleukins, lymphokines, or tumor necrosis factors. Examples of cytokines can include, for example, IL-2, IL-4, IL-6, IL-7, IL-10, IL-12, IL-15, IL-17, IL-21, CD40 L (CD154), lymphotoxin (LT, TNFβ), interferon-α, interferon-β, interferon-γ, C-CSF, GM-CSF, or M-CSF. In some embodiments, other cytokines can have altered expression. In some embodiments, the cytokine can be a marker of a non-differentiated cell. In some embodiments, the cytokine can be a marker of a differentiated cell.

Alteration of expression of a cytokine can comprise an increase or decrease in expression. In some embodiments, expression of a cytokine can be increased by at least about 5%, at least about 10%, at least about 20%, at least about 30%, at least about 40%, at least about 50%, at least about 100%, at least about 200%, at least about 300%, at least about 400%, at least about 500%, or at least about 1000% in the cultured PBMC compared with expression of a same cytokine in a cell cultured at a control culturing condition. In some embodiments, expression of a cytokine can be decreased by at least about 5%, at least about 10%, at least about 20%, at least about 30%, at least about 40%, at least about 50%, at least about 100%, at least about 200%, at least about 300%, at least about 400%, at least about 500%, or at least about 1000% in the cultured PBMC compared with expression of a same cytokine in a cell cultured at a control culturing condition.

In some embodiments, altered cytokine expression can comprise altered gene expression, such as an increase or decrease in expression of mRNA that codes for a cytokine. Altered expression of mRNA can for example comprise increased or decreased transcription of DNA to mRNA. In some cases, altered expression of mRNA can comprise increased or decreased degradation of mRNA coding for a cytokine.

Gene expression can be determined by any acceptable method. For example, gene expression can be determined using ISH, FISH, northern blot, PCR, RT-PCR, q-PCR, p-RT-PCR, or another method. RNA expression can be determined as an absolute expression (e.g., number of mRNA transcripts per cell) or a relative expression (e.g., number of mRNA transcripts compared with the number of mRNA transcripts of a housekeeping gene in the same cell, or a number of mRNA transcripts compared with the number of same mRNA transcripts of a cell cultured under control conditions).

In some embodiments, altered cytokine expression can comprise altered protein expression, such as an increase or decrease in cytokine produced or detected. Altered protein expression can for example comprise increased or decreased translation of mRNA to protein. In some cases, altered protein expression can comprise increased or decreased degradation or inactivation of the protein.

Protein expression can be determined by any acceptable method. For example, protein expression can be determined by immunoassay (e.g., ELISA), western blot, dot blot, chromatography, spectrophotometry, or another method. Protein expression can be determined as an absolute expression (e.g., number of protein molecules per cell) or a relative expression (e.g., number of protein molecules compared with the number of protein molecules of a housekeeping protein in the same cell, or number of protein molecules compared with the number of same protein molecules of a cell cultured under control conditions).

In some embodiments, the cytokine can be IL-10. In some such embodiments, the expression of IL-10 can be decreased. In some embodiments, the cytokine can be TNF-α. In some such embodiments, the expression of TNF-α can be increased. In some embodiments, the cytokine can be IL-6. In some such embodiments, the expression of IL-6 can be decreased. In some embodiments, the cytokine can comprise IFN-γ. In some such embodiments, the expression of IFN-γ can be decreased. In some embodiments, the cytokine can be TGF-β1, and the expression of TGF-β1 can be increased.

A method for determining efficacy of an anti-cancer agent can further comprise contacting a tumor cell with the PBMC and the anti-cancer agent.

An anti-cancer agent can be an agent, such as a therapeutic, that can be effective in the treatment of malignant or cancerous disease. In some embodiments, an anti-cancer agent can be effective in treating a tumor, for example inhibiting growth of tumor cells or killing tumor cells. Examples of anti-cancer agents can include alkylating agents, antimetabolites, natural products, hormones, or other agents. In some embodiments, an anti-cancer agent can be a chemotherapy drug or combination of chemotherapy drugs. In some embodiments, the anti-cancer agent can comprise a drug that can be clinically effective in reducing tumor burden, preventing or eliminating metastases, killing tumor cells, or preventing growth of tumor cells. In some embodiments, an anti-cancer agent can be a PD1 inhibitor. Examples of a PD1 inhibitor can include, for example, pembrolizumab or nivolumab.

In some embodiments, contacting the tumor cell with the PBMC and the anti-cancer agent can be performed in vitro. In some such embodiments, for example, a tumor cell can be incubated with the PBMC and the anti-cancer agent in a cell culture medium. The anti-cancer agent can be included at a concentration that is therapeutic when the anti-cancer agent is applied alone, or at a concentration that is sub-therapeutic when the anti-cancer agent is applied alone.

The PBMC and the anti-cancer agent can be applied simultaneously or one after another. In some embodiments, the tumor cell can be contacted by the PBMC before the anti-cancer agent. In some embodiments, the tumor cell can be contacted by the anti-cancer agent before the PBMC.

The tumor cell can be incubated with the PBMC and the anti-cancer agent for a time period. In some embodiments, the tumor cell can be incubated with the PBMC for at least about 1 minute, at least about 5 minutes, at least about 15 minutes, at least about 30 minutes, at least about 1 hour, at least about 2 hours, at least about 3 hours, at least about 6 hours, or at least about 12 hours, at least about 1 day, at least about 2 days, at least about 3 days, at least about 4 days, at least about 5 days, at least about 6 days, at least about 7 days, at least about 8 days, at least about 9 days, or at least about 10 days. In some embodiments, the tumor cell can be incubated with the PBMC and the anti-cancer agent for no more than about 1 minute, no more than about 5 minutes, no more than about 15 minutes, no more than about 30 minutes, no more than about 1 hour, no more than about 2 hours, no more than about 3 hours, no more than about 6 hours, no more than about 12 hours, no more than about 1 day, no more than about 2 days, no more than about 3 days, no more than about 4 days, no more than about 5 days, no more than about 6 days, no more than about 7 days, no more than about 8 days, no more than about 9 days, or no more than about 10 days. In some embodiments, the tumor cell can be incubated with the PBMC and the anti-cancer agent for about 1 minute, about 5 minutes, about 15 minutes, about 30 minutes, about 1 hour, about 2 hours, about 3 hours, about 6 hours, about 12 hours, about 1 day, about 2 days, about 3 days, about 4 days, about 5 days, about 6 days, about 7 days, about 8 days, about 9 days, about 10 days, or a range between any two foregoing values.

As a control, additional tumor cells can be incubated with a PBMC without an anti-cancer agent, with an anti-cancer agent without a PBMC, or without an anticancer agent without a PBMC.

A tumor cell or environment of the tumor cell can be analyzed to determine cytotoxicity. In some embodiments, for example, the cell culture medium can be analyzed to determine the presence of cytoplasmic markers that can indicate a loss of membrane integrity and thus a dead cell, as described above. In some embodiments, a cytoplasmic marker can be a naturally existing marker, such as an enzyme, or can be introduced artificially, such as a radioactive or fluorescent marker loaded into the cells prior to exposure to PBMCs and/or the anti-cancer agent.

In some embodiments, contacting the tumor cell with the PBMC and the anti-cancer agent can be performed in vivo. In some such embodiments, the PBMC and the anti-cancer agent can be administered to a subject having a tumor cell, such as a subject having a tumor or a subject having a metastasis. The subject can be a human subject or a laboratory animal (e.g., a mouse, a rat, a guinea pig, a rabbit, a dog, or a cat). Administering the PBMC and anti-cancer agent to the subject can be performed as described above.

A method for determining efficacy of an anti-cancer agent can further comprise measuring the cytotoxicity against the tumor cell, thereby determining the efficacy of the ant-cancer agent against the tumor cell. In some embodiments, the cytotoxicity can be measured after the time of incubation, as described above. For example, the cytotoxicity can be measured after at least about five days.

In some embodiments, cytotoxicity of the anti-cancer agent can be measured by assessing the tumor burden of the subject after administration. In some embodiments, the subject can have a smaller tumor, increased tumor cell death, or fewer tumor cells after administration of the anti-cancer agent and PBMC.

In some embodiments, cytotoxicity of the anti-cancer agent with the PBMC can be increased relative to the cytotoxicity of the anti-cancer agent against the tumor cell when the anti-cancer agent is contacted to the tumor cell during the control condition. The control condition can be an in vitro condition (e.g., a culturing condition) or an in vitro condition (e.g., in a subject). In some embodiments, the cytotoxicity can be increased by at least about 5%, at least about 10%, at least about 15%, at least about 20%, at least about 25%, at least about 30%, at least about 35%, at least about 40%, at least about 45%, or at least about 50% relative to a cytotoxicity of the anti-cancer agent against the tumor cell, when the anti-cancer agent is contacted to the tumor cell at the control culturing condition.

### Methods for Enriching a Cell Subpopulation.

Also provided herein are methods for enriching a cell subpopulation from a source population of cells. In some embodiments, a method of enriching a cell subpopulation can comprise culturing a pan T cell under conditions provided above, wherein the cell subpopulation comprises CD8+ cells.

Also provided herein are methods for enriching a cell subpopulation from a source population of cells. In some embodiments, a method of enriching a cell subpopulation can comprise culturing a pan T cell under conditions provided above, wherein the cell subpopulation comprises CD4+ cells.

In some embodiments, the cell subpopulation can be cultured for example at from about 1% to about 15% oxygen, and a pressure condition of no more than about 2 PSI above atmospheric pressure. In some such embodiments, the oxygen level can be 15%, and the pressure condition can be 2 PSI over atmospheric pressure.

### Methods for Increasing a Cell Volume.

Also provided herein are methods for increasing the cell volume of an immune cell. Methods for increasing a cell volume of an immune cell can comprise culturing the immune cell, wherein the cell volume of the immune cell is increased relative to an immune cell cultured under a control condition.

In some embodiments, the oxygen level during culturing can be regulated to a hypoxic level with respect to the ambient oxygen level. In some embodiments, the total atmospheric pressure can be regulated to a hyperbaric level with respect to the ambient atmospheric pressure. In some embodiments of the method, the oxygen level can be regulated to a hypoxic level with respect to the ambient oxygen level, and the total atmospheric pressure is regulated to a hyperbaric level with respect to the ambient atmospheric pressure. In embodiments, the atmospheric pressure and the oxygen level are regulated independently of each other such that a hypoxic oxygen level prevails in spite of an overall hyperbaric condition.

In some embodiments, the immune cell can be cultured for example at from about 1% to about 15% oxygen, and a pressure condition of no more than about 2 PSI above atmospheric pressure. In some such embodiments, the oxygen level can be 15%, and the pressure condition can be 2 PSI above atmospheric pressure.

In some embodiments, the oxygen level can be about 1%, about 2%, about 3%, about 4%, about 5%, or a range between any two foregoing values. In some embodiments, the oxygen level can be 1% to about 5% oxygen.

In some embodiments, the immune cell can be a T cell. In some embodiments, the immune cell can be a B cell. In some embodiments, the immune cell can be another type of cell.

Control culturing conditions can comprise standard culturing conditions. In some embodiments, control culturing conditions can comprise an ambient (e.g., of the room or atmospheric) oxygen level or pressure condition.

A control culturing condition can comprise an oxygen level of about 15%, about 16%, about 17%, about 18%, about 19%, or about 20%, or a range between any two foregoing values. In some embodiments, a control culturing condition can comprise an oxygen level of at least about 15%, at least about 16%, at least about 17%, at least about 18%, at least about 19%, or at least about 20%.

A control culturing condition can comprise a pressure condition of about 0 PSI above atmospheric pressure, about 0.5 PSI above atmospheric pressure, about 1 PSI above atmospheric pressure, or a range between any two foregoing values. In some embodiments, for example, a control culturing condition can comprise a pressure condition of about 0 PSI above atmospheric pressure. In some embodiments, a control culturing condition can comprise a pressure condition of no more than 0.5 PSI above atmospheric pressure or no more than 1 PSI above atmospheric pressure.

In some embodiments, a control culturing condition can comprise a combination of a given oxygen level and a given pressure condition, such as an oxygen level and pressure condition of a control culturing condition provided above. For example, in some embodiments, control culturing condition can comprise about 18% oxygen and a pressure condition of 0 PSI above atmospheric pressure.

In some embodiments, the cell volume can be increased by at least 10 cubic microns (μ³), at least 50μ³, at least 100μ³, at least 150μ³, or at least 200μ³, or a range between any two foregoing values. In some embodiments, the cell volume can be increased by at least 100μ³.

### Cell Populations.

The source cell population can include, for example, immune cells, tumor cells, non-cancer cells, and stem cells. The immune cell populations can include, for example, monocytes, macrophages, antigen-presenting cells, dendritic cells, monocytes, macrophages, pan T-cells, T-cells, tumor-infiltrating T cells, regulatory T cells, natural killer (NK) cells, neutrophils, and B-cells. Stem cells can include, for example, naturally occurring stem cells or induced stem cells. Stem cell populations may include mesenchymal stem cells, these stem cell populations including any of progenitor, immature, and mature subgroups.

### Culture Conditions.

As described herein, oxygen levels can be referred to in terms of a concentration % value, i.e., the relative amount of oxygen present with respect to all gases present within a given volume, regardless of the summed total atmospheric pressure of all gases present.

In addition of regulating levels of oxygen and total gas pressure, some embodiments of methods of modulating phenotype through atmospheric means may include regulating temperature and regulating pH. Regulating pH within cell culture media with a bicarbonate buffering system is typically done by regulating the concentration of carbon dioxide in the internal atmosphere. Regulation of pH within cell culture media can also be done by way of using other buffering systems.

“PSI”, as used herein, refers to pressure (pounds per square inch) over the ambient atmospheric pressure.

A term that incorporates both variables, the O-P condition; refers to a condition that is defined by the combination of the two variable atmospheric parameters (oxygen level and total gas pressure). Any terminology that defines each parameter, respectively can be used to identify the O-P condition. For example, oxygen level may be defined in terms of concentration (relative % of total gas) or partial pressure (absolute level of oxygen per unit volume). By way of a specific example, an O-P condition could be expressed as “3% oxygen—3PSI”.

Potency-level phenotype can refer to a phenotypic spectrum that ranges from the totipotency to a terminally differentiated cell. Other cell potency level phenotypes include, for example, pluripotency, multipotency, oligopotency, and unipotency.

### Applications Related to Cell Differentiation.

In some embodiments, the present disclosure provides a method to identify atmospheric condition parameter settings (such as an oxygen level and total atmospheric pressure level) to maintain an erythrocyte differentiation state for a period of time with minimal differentiation drift. In some embodiments, the oxygen level can be between about 1% to about 15%. In some embodiments, the pressure condition can be no more than about 2 PSI. Erythrocytes can be derived from differentiation of, for example, a hematopoietic stem cell. Then, the differentiated hematopoietic stem cell can proceed through a common myeloid progenitor cell stage, then to a megakaryocyte-erythroid progenitor cell stage, and finally, to the erythrocyte stage.

In some embodiments, the present disclosure provides a method to identify atmospheric condition parameters (such as an oxygen level and total atmospheric pressure level) that favor the differentiation of monocytes into a M1-polarized macrophage (in contrast to a M2-polarized macrophage). In some embodiments, the present disclosure provides a method to identify atmospheric condition parameters that favor differentiation of monocytes toward an M1-polarized macrophage population. In some embodiments, the oxygen level can be between about 1% to about 15%. In some embodiments, the pressure condition can be no more than about 2 PSI.

M1-polarization of macrophages can occur by bacterial lipopolysaccharides, as well as cytokines such as GM-CSF or interferons. M1-activation of macrophages can cause phenotypic changes such as release of pro-inflammatory cytokines, reactive oxygen species, and nitrogen radicals, and M1-activation can increase the level of attack on targeted bacteria.

Macrophages can be derived through differentiation of a hematopoietic stem cell, and can proceed through a common myeloid progenitor cell stage, then a granulocyte-macrophage progenitor cell state, then to a monocyte stage, and finally the macrophage stage.

In some embodiments of the method, oxygen is at a hypoxic level and total atmospheric pressure is hyperbaric.

In some embodiments, the present disclosure provides a method of increasing a rate of differentiation of mouse mature adipocytes from pre-adipocytes under optimal atmospheric conditions as compared to ambient atmospheric conditions. In some embodiments, oxygen is at a hypoxic level and total atmospheric pressure is hyperbaric. In some embodiments, the present disclosure provides a method of increasing a rate of differentiation of neurons from iPSCs under optimal atmospheric conditions as compared to standard or ambient atmospheric conditions. In some embodiments, oxygen is at a hypoxic level and total atmospheric pressure is hyperbaric.

In some embodiments, the present disclosure provides a method to identify atmospheric condition parameters (such as an oxygen level and total atmospheric pressure level) that are optimal for driving differentiation from iPSC to hematopoietic stem cell (HSC) population, and then to a megakaryocyte population. The presence of the various phenotypes can be determined by, for example, FACS analysis.

In some embodiments, the present disclosure provides a method for increasing the growth and differentiation of cardiomyocytes from iSPCs under optimal atmospheric conditions as compared to culturing under standard or ambient conditions.

In some embodiments, the present disclosure provides a method to identify atmospheric condition parameters (such as an oxygen level and total atmospheric pressure level) that are optimal for hypoxia inducible factor-1 (HIF-1) induction in prostate cancer cell line PC-3. In some embodiments, the present disclosure provides a method to determine a time course of induction with and without increased atmospheric pressure for induction of HIF-1 in, for example, a prostate cancer cell line. The time course of induction under ambient atmospheric pressure condition can peak at about 24 hours, and in the presence of 2 PSI over ambient pressure, the HIF 1 can peak at 6 hours.

In some embodiments, the present disclosure provides a method for improving the development of pancreatic cancer cell organoids from biopsies under optimal atmospheric conditions compared to culturing under standard or ambient conditions.

In some embodiments, the present disclosure provides a method to identify atmospheric condition parameters (such as an oxygen level and total atmospheric pressure level) that are optimal for generating ovarian cancer clusters (tumor organoids). These tumor organoids can then be used, for example, as a substrate for testing efficacy of chemotherapeutic drugs.

In some embodiments, the present disclosure provides a method to identify atmospheric condition parameters (such as an oxygen level and total atmospheric pressure level) for culturing of biopsy samples of tumors. The resulting cell population can then be used as a substrate for testing anti-cancer drugs or candidate drugs. For example, colorectal cancer cells from cell lines can be grown as spheroids, and then the killing of the colorectal cancer cell spheroids can be activated by T or NK cells, and the killing can be observed under optimal atmospheric conditions. In another example, B16 melanoma cell lines can grow as spheroids and CD8-splenocyte mediated killing can be observed.

In some embodiments, the present disclosure provides a method to identify atmospheric condition parameters (such as an oxygen level and total atmospheric pressure level) that modulate the in vitro growth rate of non-small cell lung cancer cells (NSCLC) derived from biopsy samples.

In some embodiments, oxygen is at a hypoxic level and total atmospheric pressure is hyperbaric as compared to an ambient condition.

In some embodiments, the present disclosure provides a method for determining the optimal atmospheric conditions for thawing cells from a frozen vial. In some embodiments, the thawing and subsequent viability of myeloid leukemia (AML) cancer cells under optimal atmospheric conditions can have an increase in viability over cells thawed under standard or ambient conditions.

Treg cells are a specialized subpopulation of T cells that act broadly to suppress the immune response, thereby maintaining homeostasis and self-tolerance. More particularly, Tregs inhibit effector T cell proliferation and cytokine production, and play a role in reducing the likelihood of autoimmune reactivity. T cells are derived from a differentiation path that originate from a hematopoietic stem cell, and proceed through a common lymphoid progenitor cell, and then finally to a T-cell stage.

Other phenotypic effects of hypoxic and high pressure conditions on Treg cells can be observed. In some embodiments, the present disclosure provides a method to identify atmospheric condition parameters (such as an oxygen level and total atmospheric pressure level) that supports growth of regulatory T cells (Tregs) at a rate faster than if the Tregs are cultured under standard incubator conditions. In some embodiments, the present disclosure provides a method of culturing maturity markers on regulatory T cells from patients with kidney inflammation under various atmospheric conditions to understand changes that occur in these cells during the course of inflammatory disease, and/or directed toward use of these cells as a substrate for testing therapeutic drug candidates.

Treg cells are included in the development of CAR-T cells (chimeric antigen receptor T cells, T-cell referring to thymus-originating lymphocytes) for immunotherapies. Viral transduction of Treg cells is a part of conferring the chimeric antigen receptor status of these cells. In some embodiments, genetic transfection, and viral transduction, can be more efficient under hypoxic and/or hyperbaric conditions.

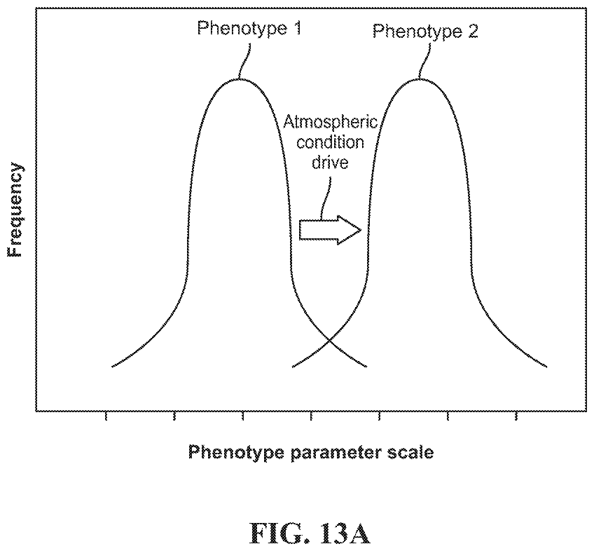

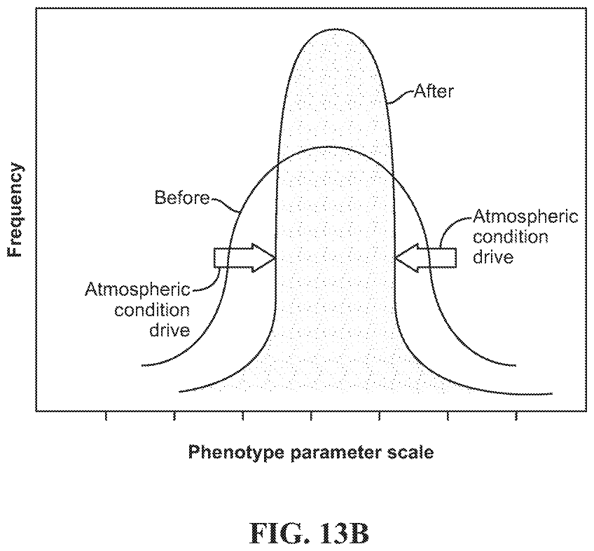

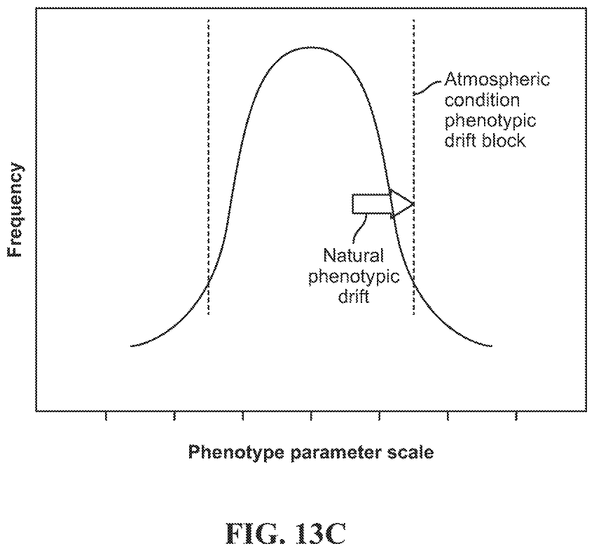

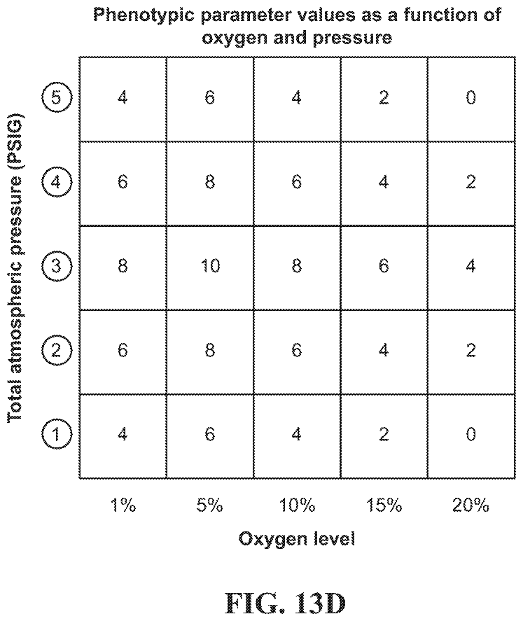

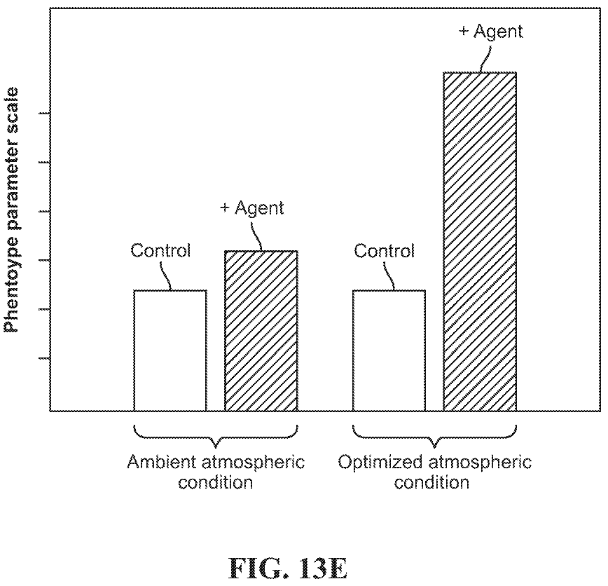

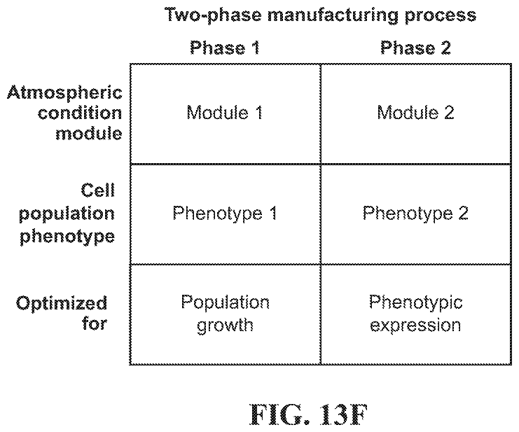

During their maturation, Treg cells progress from a naïve state to a more specific antigen-targeted state. As raw material or a substrate for a transduction process to become a chimeric antigen equipped Treg cell, naïve cells (rather than already antigen-specified cells) can be advantageous. A culture condition described here can stabilize the developmental or maturational state of Treg cells in their naïve state. Such a stabilization of phenotype is schematically-depicted in FIG. 13C.

In some embodiments, the present disclosure provides a method to identify atmospheric condition parameters (such as an oxygen level and total atmospheric pressure level) that are optimal for sustaining T cell function. T-cell function can be eroded by T cell exhaustion. T cell exhaustion involves a number of dysfunctions, including a progressive loss of effector function due to prolonged antigen stimulation of the cell, as may occur in the body during chronic infection or the long-term presence of a cancer. Other factors in the environment, such as an imbalance in the normal cytokine profiles, can also contribute to exhaustion. T cell exhaustion can affect cells that have been taken from a patient for use in a clinical manufacturing process, such as creating CAR-T cells, and T-cell exhaustion can continue or occur during a manufacturing process. Thus, a well-controlled and consistent in vitro environment, including atmospheric variables such as oxygen level and total gas pressure levels can contribute to the development of robust T cell populations that are not-exhausted, or which have recovered from exhaustion. In some embodiments, oxygen is at a hypoxic level and total atmospheric pressure is hyperbaric as compared to ambient conditions.

In some embodiments, the present disclosure provides a method to identify atmospheric condition parameters (such as an oxygen level and total atmospheric pressure level) that are optimal for the killing of cancer cells by Natural Killer cells. NK92 is a Natural Killer cell line derived from human patient with non-Hodgkin's lymphoma that is constitutively activated and lacks inhibitory receptors, making NK92 a potential off-the-shelf cell therapy candidate.

In some embodiments, the present disclosure provides a method for improving the long-term maintenance of tumor infiltrating lymphocytes (TILs) phenotype during culture under optimal atmospheric conditions as compared to culturing under standard or ambient conditions.

In some embodiments, the present disclosure provides a method to identify atmospheric condition parameters (such as an oxygen level and total atmospheric pressure level) that are optimal for proliferating chimeric antigen receptor-T cells (CAR-T) in vitro. The chimeric cell receptors of CAR-T cells are genetically engineered to bind to specific antigens, such as those that are expressed on the surface of cancer cells, and to activate the T cell cells once the T cells encounter a cancer cell expressing the antigen. In terms of evaluating the effectiveness of such CAR-T cells, the variables include the effectiveness of the engineered receptor in recognizing the target antigen and the effectiveness of activating the T cells. Thus, large amounts of robust and consistent-quality T cells are needed for screening these receptors and their efficacy.

In some embodiments, oxygen is at a hypoxic level and total atmospheric pressure is hyperbaric as compared to ambient conditions.

### Pharmaceutical Compositions and Dosing.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, Ed., Mack Publishing Co., Easton, Pa., 1990; and Remington, The Science and Practice of Pharmacy 20th Ed. Mack Publishing, 2000).

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosacchandes, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration may be sterilized. This may be accomplished by, for example, filtration through sterile filtration membranes, or any other art-recognized method for sterilization. PBMC compositions are generally placed into a container having a sterile access port, for example, an intravenous solution bag or vial having a stopper pierceable by a hypodermic injection needle. Other methods for sterilization and filtration are known in the art and are contemplated herein.

In some embodiments, the compositions can be formulated to be free of pyrogens such that they are acceptable for administration to a subject. Testing compositions for pyrogens and preparing pharmaceutical compositions free of pyrogens are well understood to one of ordinary skill in the art.

The compositions according to the present invention may be in unit dosage forms such as solutions or suspensions, tablets, pills, capsules, powders, granules, or suppositories, etc., for intravenous, oral, parenteral or rectal administration, or administration by inhalation or insufflation.

The phrase “pharmaceutically acceptable” refers to molecular entities and compositions that are physiologically tolerable and do not typically produce an allergic or similar untoward reaction, such as gastric upset, dizziness and the like, when administered to a subject.

A “unit dose” when used in reference to a therapeutic composition or pharmaceutical composition refers to physically discrete units suitable as unitary dosage for humans, each unit containing a predetermined quantity of active material calculated to produce the desired therapeutic effect in association with the required diluent; i.e., carrier, or vehicle.

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Additionally, continuous intravenous infusion sufficient to maintain concentrations in the blood are contemplated.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” as used herein, generally refers to a range that is 2%, 5%, 10%, 15% greater than or less than (±) a stated numerical value within the context of the particular usage. For example, “about 10” would include a range from 8.5 to 11.5. As used herein, the terms “about” and “approximately,” when used to modify a numeric value or numeric range, indicate that deviations of up to about 0.2%, about 0.5%, about 1%, about 2%, about 5%, about 7.5%, or about 10% (or any integer between about 1% and 10%) above or below the value or range remain within the intended meaning of the recited value or range.

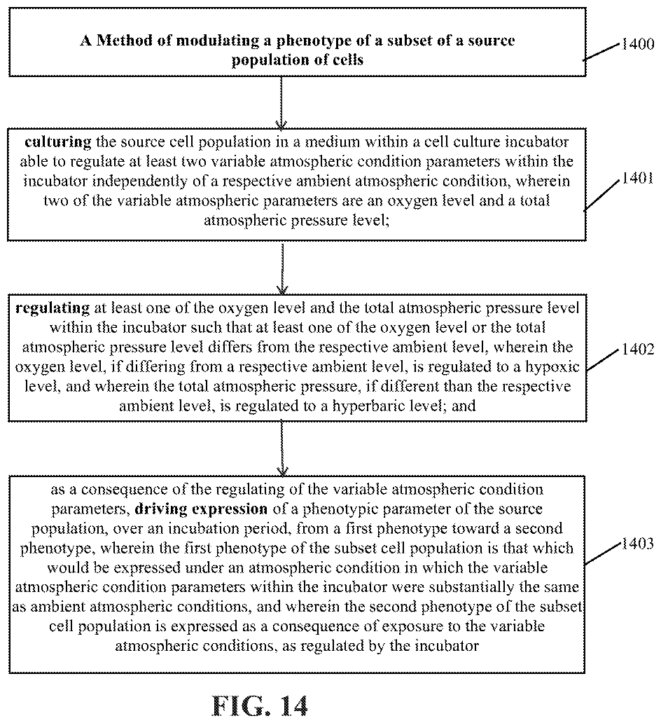

Embodiments of method 1400 (FIG. 14) include culturing the source cell population in a medium within a cell culture incubator able to regulate at least two variable atmospheric condition parameters within the incubator independently of a respective ambient atmospheric condition, wherein two of the variable atmospheric parameters are an oxygen level and a total atmospheric pressure level 1401; regulating at least one of the oxygen level and the total atmospheric pressure level within the incubator such that at least one of the oxygen level or the total atmospheric pressure level differs from the respective ambient level, wherein the oxygen level, if differing from a respective ambient level, is regulated to a hypoxic level, and wherein the total atmospheric pressure, if different than the respective ambient level, is regulated to a hyperbaric level; 1402 and as a consequence of the regulating of the variable atmospheric condition parameters, driving expression of a phenotypic parameter of the source population, over an incubation period, from a first phenotype toward a second phenotype, wherein the first phenotype of the subset cell population is that which would be expressed under an atmospheric condition in which the variable atmospheric condition parameters within the incubator were substantially the same as ambient atmospheric conditions, and wherein the second phenotype of the subset cell population is expressed as a consequence of exposure to the variable atmospheric conditions, as regulated by the incubator 1403.

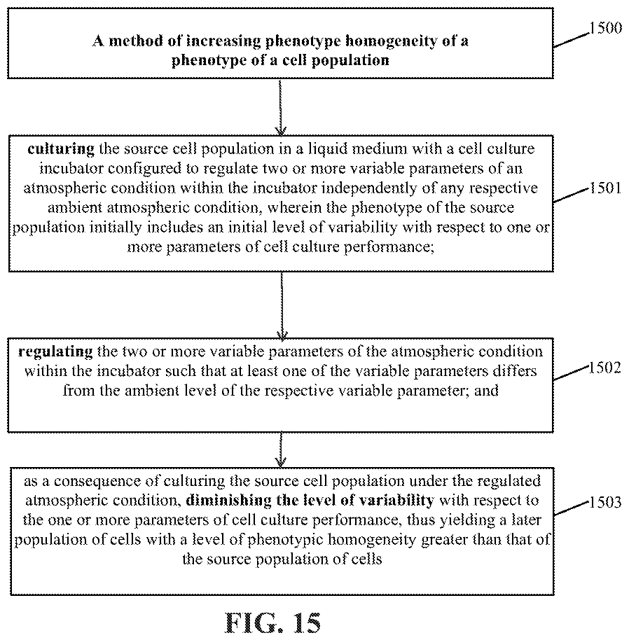

Embodiments of method 1500 (FIG. 15) include culturing the source cell population in a liquid medium within a cell culture incubator configured to regulate two or more variable parameters of an atmospheric condition within the incubator independently of any respective ambient atmospheric condition, wherein the phenotype of the source population initially includes an initial level of variability with respect to one or more parameters of cell culture performance 1501; regulating the two or more variable parameters of the atmospheric condition within the incubator such that at least one of the variable parameters differs from the ambient level of the respective variable parameter 1502; and as a consequence of culturing the source cell population under the regulated atmospheric condition, diminishing the level of variability with respect to the one or more parameters of cell culture performance, thus yielding a later population of cells with a level of phenotypic homogeneity greater than that of the source population of cells. 1503

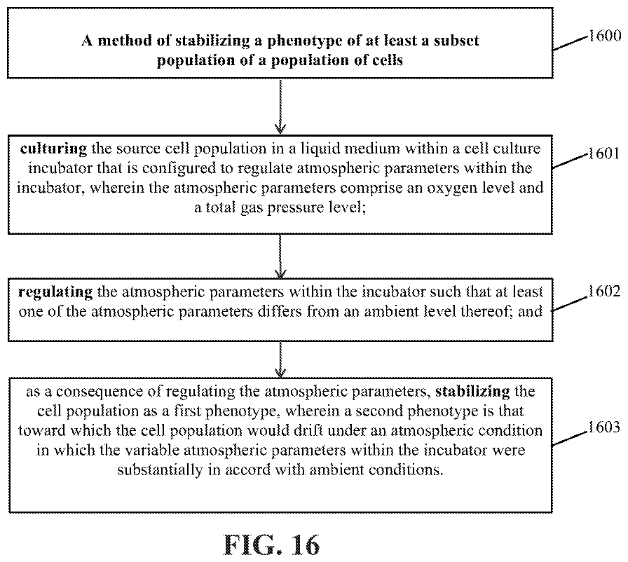

Embodiments of method 1600 (FIG. 16) include culturing the source cell population in a liquid medium within a cell culture incubator that is configured to regulate atmospheric parameters within the incubator, wherein the atmospheric parameters comprise an oxygen level and a total gas pressure level 1601; regulating the atmospheric parameters within the incubator such that at least one of the atmospheric parameters differs from an ambient level thereof 1602; and as a consequence of regulating the atmospheric parameters, stabilizing the cell population as a first phenotype, wherein a second phenotype is that toward which the cell population would drift under an atmospheric condition in which the variable atmospheric parameters within the incubator were substantially in accord with ambient conditions. 1603

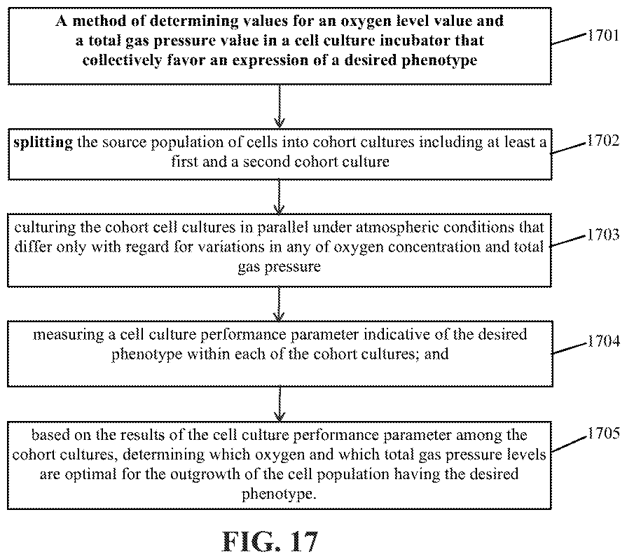

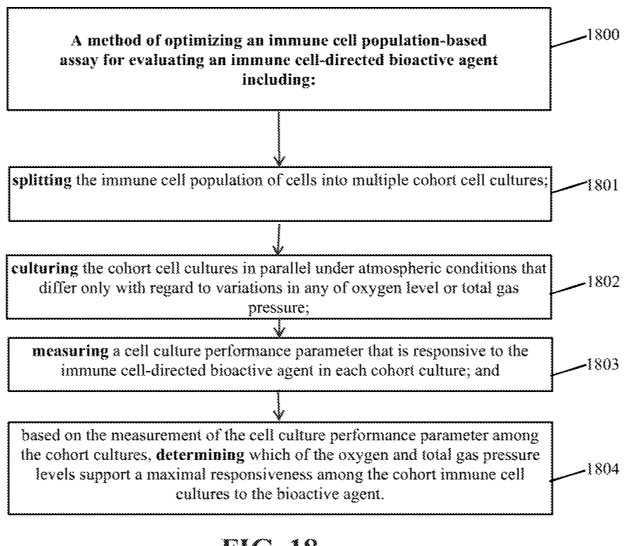

Embodiments of method 1700 (FIG. 17) include splitting the source population of cells into cohort cultures including at least a first and a second cohort culture 1701; culturing the cohort cell cultures in parallel under atmospheric conditions that differ only with regard for variations in any of oxygen concentration and total gas pressure 1702; measuring a cell culture performance parameter indicative of the desired phenotype within each of the cohort cultures 1703; and based on the results of the cell culture performance parameter among the cohort cultures, determining which oxygen and which total gas pressure levels are optimal for the outgrowth of the cell population having the desired phenotype 1704.

Embodiments of method 1800 (FIG. 18) include splitting the immune cell population of cells into multiple cohort cell cultures 1801; and culturing the cohort cell cultures in parallel under atmospheric conditions that differ only with regard to variations in any of oxygen level or total gas pressure 1802; and measuring a cell culture performance parameter that is responsive to the immune cell-directed bioactive agent in each cohort culture 1803; and based on the measurement of the cell culture performance parameter among the cohort cultures, determining which of the oxygen and total gas pressure levels support a maximal responsiveness among the cohort immune cell cultures to the bioactive agent. 1804

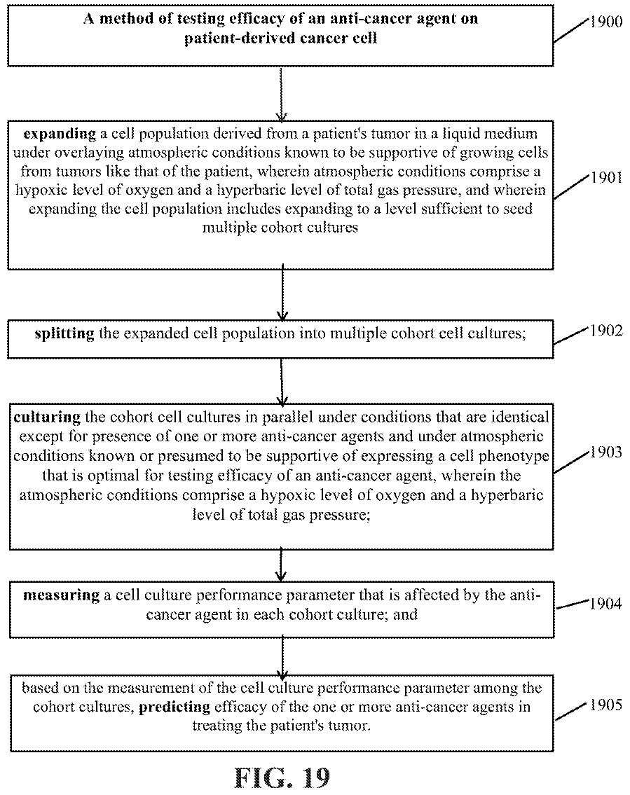

Embodiments of method 1900 (FIG. 19) include expanding a cell population derived from a patient's tumor in a liquid medium under overlaying atmospheric conditions known to be supportive of growing cells from tumors like that of the patient, wherein atmospheric conditions comprise a hypoxic level of oxygen and a hyperbaric level of total gas pressure, and wherein expanding the cell population includes expanding to a level sufficient to seed multiple cohort cultures 1901; splitting the expanded cell population into multiple cohort cell cultures 1902; culturing the cohort cell cultures in parallel under conditions that are identical except for presence of one or more anti-cancer agents and under atmospheric conditions known or presumed to be supportive of expressing a cell phenotype that is optimal for testing efficacy of an anti-cancer agent, wherein the atmospheric conditions comprise a hypoxic level of oxygen and a hyperbaric level of total gas pressure 1903; measuring a cell culture performance parameter that is affected by the anti-cancer agent in each cohort culture 1904; and based on the measurement of the cell culture performance parameter among the cohort cultures, predicting efficacy of the one or more anti-cancer agents in treating the patient's tumor 1905.

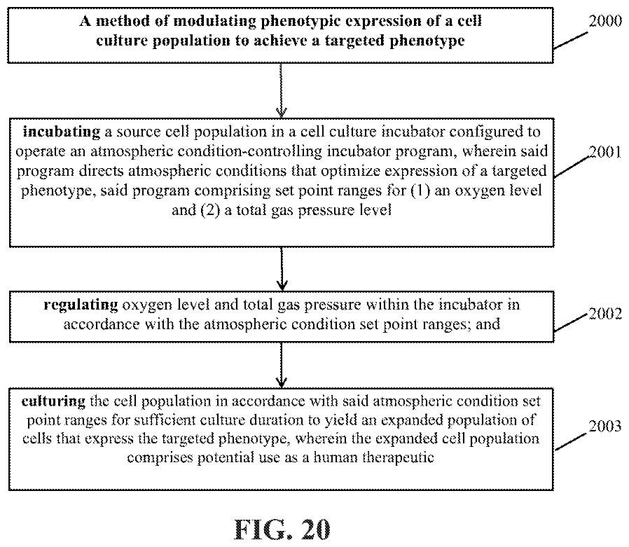

Embodiments of method 2000 (FIG. 20) include incubating a source cell population in a cell culture incubator configured to operate an atmospheric condition-controlling incubator program, wherein said program directs atmospheric conditions that optimize expression of a targeted phenotype, said program comprising set point ranges for (1) an oxygen level and (2) a total gas pressure level 2001; regulating oxygen level and total gas pressure within the incubator in accordance with the atmospheric condition set point ranges 2002; and culturing the cell population in accordance with said atmospheric condition set point ranges for sufficient culture duration to yield an expanded population of cells that express the targeted phenotype, wherein the expanded cell population comprises potential use as a human therapeutic 2003.

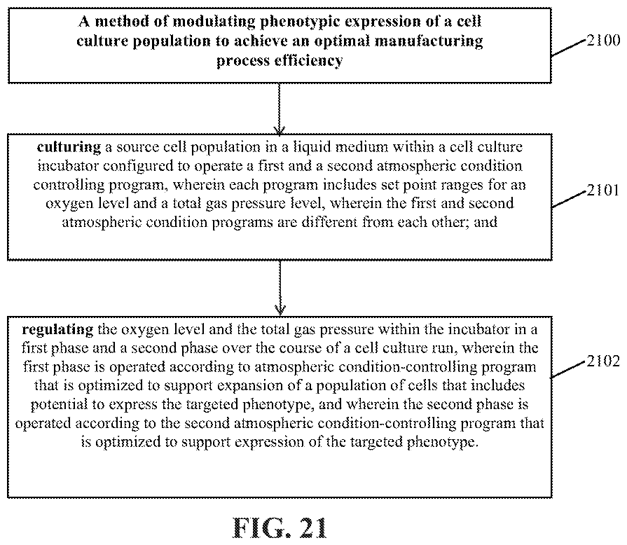

Embodiments of method 2100 (FIG. 21) include culturing a source cell population in a liquid medium within a cell culture incubator configured to operate a first and a second atmospheric condition-controlling program, wherein each program includes set point ranges for an oxygen level and a total gas pressure level, wherein the first and second atmospheric condition programs are different from each other 2101; and regulating the oxygen level and the total gas pressure within the incubator in a first phase and a second phase over the course of a cell culture run, wherein the first phase is operated according to atmospheric condition-controlling program that is optimized to support expansion of a population of cells that includes potential to express the targeted phenotype, and wherein the second phase is operated according to the second atmospheric condition-controlling program that is optimized to support expression of the targeted phenotype 2102.

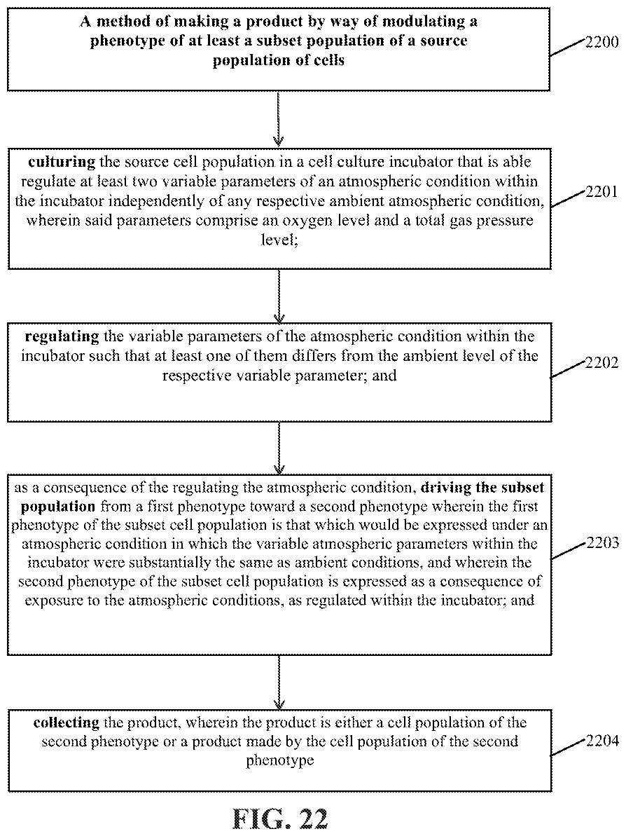

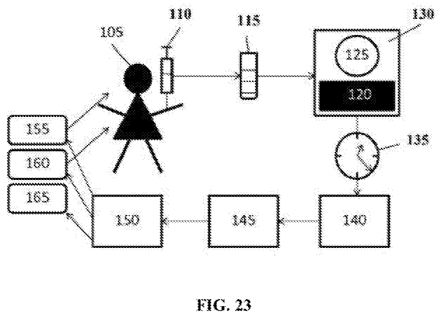

Embodiments of method 2200 (FIG. 22) include culturing the source cell population in a cell culture incubator that is able regulate at least two variable parameters of an atmospheric condition within the incubator independently of any respective ambient atmospheric condition, wherein said parameters comprise an oxygen level and a total gas pressure level 2201; regulating the variable parameters of the atmospheric condition within the incubator such that at least one of them differs from the ambient level of the respective variable parameter 2202; and as a consequence of the regulating the atmospheric condition, driving the subset population from a first phenotype toward a second phenotype wherein the first phenotype of the subset cell population is that which would be expressed under an atmospheric condition in which the variable atmospheric parameters within the incubator were substantially the same as ambient conditions, and wherein the second phenotype of the subset cell population is expressed as a consequence of exposure to the atmospheric conditions, as regulated within the incubator 2203; and collecting the product, wherein the product is either a cell population of the second phenotype or a product made by the cell population of the second phenotype 2204.

### EXAMPLES

### Example 1: Effects of Hypoxic and Hyperbaric Culture Conditions on Cytokine Secretion

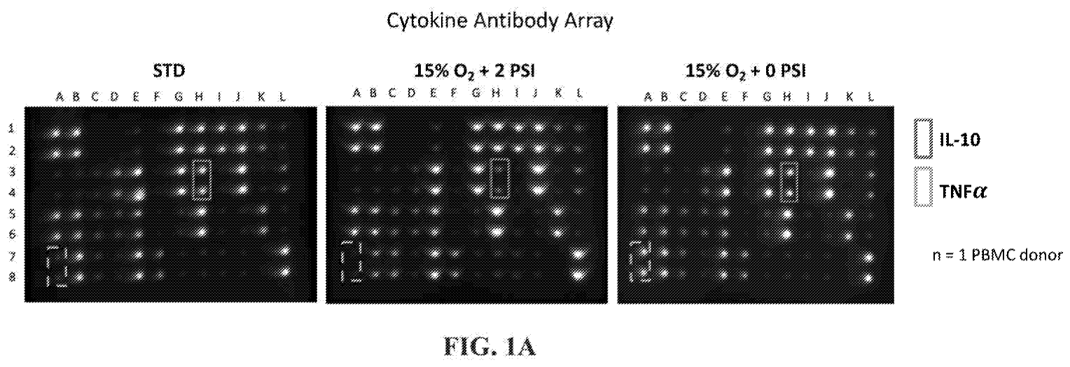

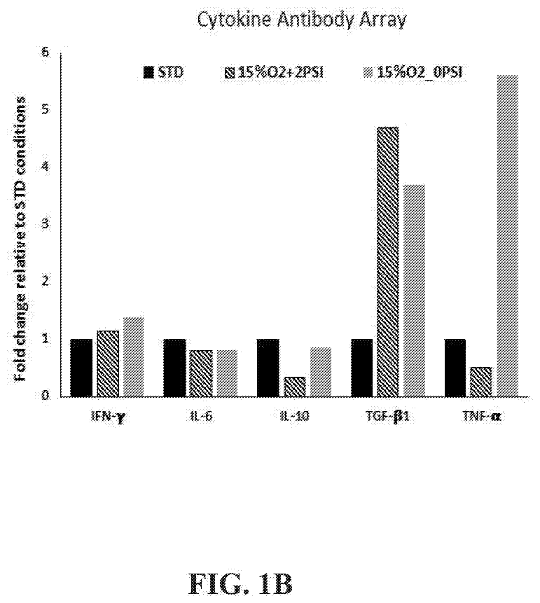

Peripheral blood mononuclear cells (PBMC) were isolated from a healthy volunteer and cultured under different hypoxic and hyperbaric conditions. PBMCs were cultured under normal atmospheric conditions (ambient conditions), hypoxic and hyperbaric conditions (2 PSI above ambient conditions, 15% O₂), and hypoxic conditions (ambient pressure, 15% O₂). All PBMCs were cultured in Immunocult-XF T cell expansion medium with IL-2 (10 ng/mL) and activated with anti-CD3/CD28 Human T-Activator Dynabeads at the beginning of the culture period. Cell culture supernatants were collected and analyzed using a human cytokine antibody array (Abcam, Cat #ab133997). Tables 1 and 2 show maps of the antibody array used, Table 1 shows cytokine identities of columns A-F of the array, and Table 2 shows columns G-L. Signals on the membrane were developed with ECL detection reagent (GE Healthcare) and imaged on a MyECL Imager system (Thermo Scientific). FIG. 1A shows images of the cytokine arrays for the three different hypoxic and hyperbaric conditions, and FIG. 1B shows a summary of selected cytokines from the array in FIG. 1A (expressed as fold change relative to standard atmospheric conditions).

As seen in FIGS. 1A and 1B, levels of secreted IL-10 were decreased in the hypoxic and hyperbaric conditions but were not affected by hypoxic conditions alone. Secreted levels of TNF-α were not affected in the hypoxic and hyperbaric conditions but were increased in the hypoxic conditions. Levels of secreted IFN-γ and IL-6 showed minimal changes under the different conditions, and levels of secreted TNF-α were increased in both hypoxic, and, hypoxic and hyperbaric, conditions.

TABLE 1
Cytokine map for cytokine antibody array in FIG. 1A, columns A-F

| | A | B | C | D | E | F |
|---|---|---|---|---|---|---|
| 1 | Pos | Pos | Neg | Neg | ENA-78 | GCSF |
| 2 | Pos | Pos | Neg | Neg | ENA-78 | GCSF |
| 3 | IL-2 | IL-3 | IL-4 | IL-5 | IL-6 | IL-7 |
| 4 | IL-2 | IL-3 | IL-4 | IL-5 | IL-6 | IL-7 |
| 5 | MCP-1 | MCP-2 | MCP-3 | MCSF | MDC | MIG |
| 6 | MCP-1 | MCP-2 | MCP-3 | MCSF | MDC | MIG |
| 7 | TNF-α | TNF-β | EGF | IGF-1 | Angionenin | Oncostatin M |
| 8 | TNF-α | TNF-β | EGF | IGF-1 | Angionenin | Oncostatin M |

TABLE 2
Cytokine map for cytokine antibody array in FIG. 1A, columns G-L

| | G | H | I | J | K | L |
|---|---|---|---|---|---|---|
| 1 | GM-CSF | GRO | GRO-α | I-309 | IL-1 α | IL-1 β |
| 2 | GM-CSF | GRO | GRO-α | I-309 | IL-1 α | IL-1 β |
| 3 | IL-8 | IL-10 | IL-12 p40/p70 | IL-13 | IL-15 | IFN-y |
| 4 | IL-8 | IL-10 | IL-12 p40/p70 | IL-13 | IL-15 | IFN-y |
| 5 | MIP-1 δ | RANTES | SCF | SDF-1 | TARC | TGF- |
| | | | | | | β1 |
| 6 | MIP-1 δ | RANTES | SCF | SDF-1 | TARC | TGF- |
| | | | | | | β1 |
| 7 | Thrombo- | VEGF | PDGF BB | Leptin | Neg | Pos |
| | poietin | | | | | |
| 8 | Thrombo- | VEGF | PDGF BB | Leptin | Neg | Pos |
| | poietin | | | | | |

### Example 2: Effects of Hypoxic and Hyperbaric Culture Conditions on Tumor Killing Activity of PBMCs, and Efficacy of Immunotherapies

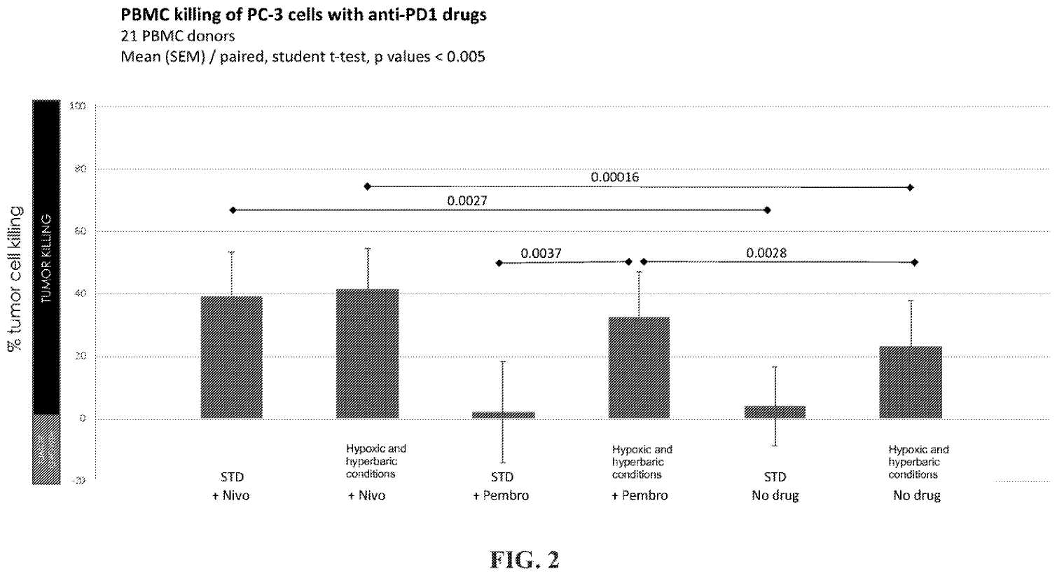

PBMCs were isolated from 21 healthy volunteers. The PBMCs were cultured together with target tumor cells, (PC-3, a prostate cancer cell line), at an effector to target ratio of 20:1. All cells were cultured in RPMI1640 medium supplemented with 10% fetal bovine serum over a 5 day period. PBMCs were not activated with IL-2. Cell viability was measured at the end of the 5 day period by CellTiter Glo Assay (Promega) with 3 or 4 technical replicates. Each of the 21 PBMC isolates, and target cells, was cultured under either ambient conditions, or hypoxic and hyperbaric conditions (1% O₂, 2 PSI above ambient pressure), and with or without anti-PD-1 antibodies. The anti-PD-1 antibodies used were Nivolumab and Pembrolizumab (10 ug/ml). Average results across the 21 different PBMC isolates are shown in FIG. 2, with error bars representing the standard error of the mean, and p values were calculated using paired Student's t tests.

As shown in FIG. 2, the hypoxic and hyperbaric conditions did not have a noticeable effect on the efficacy of Nivolumab treatment, but Pembrolizumab treatment was more efficient at activating PBMCs to kill tumor cells under hypoxic and hyperbaric conditions (p value of 0.0037).

In the absence of drug treatment hypoxic and hyperbaric conditions showed a trend towards increasing efficacy of tumor cell killing; however, the results did not reach statistical significance, perhaps due to variation among donors.

### Example 3: Effects of Hypoxic and Hyperbaric Culture Conditions on T Cell Expansion

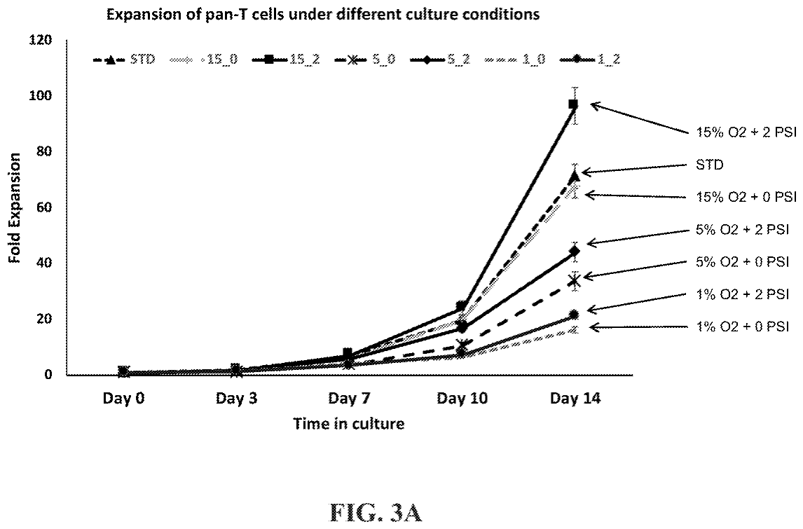

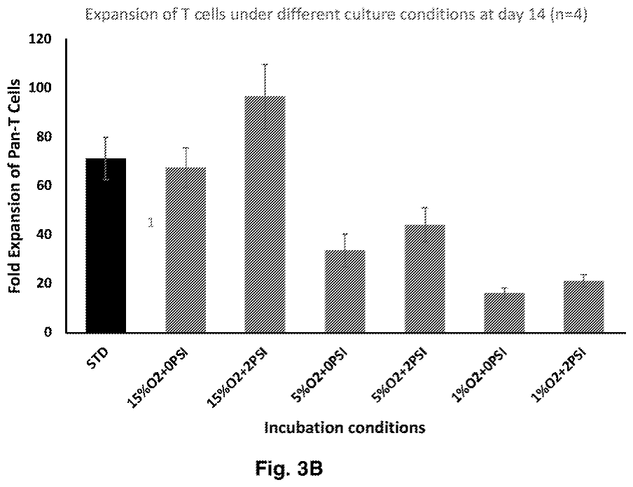

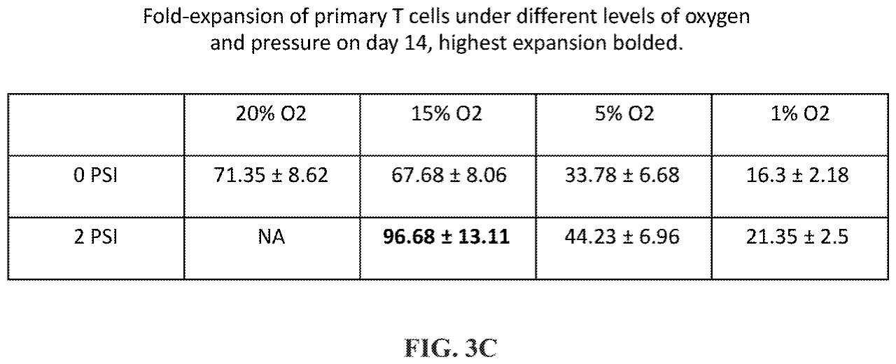

PBMCs were isolated from 4 volunteers. T cells were isolated from the PBMCs using a human Pan-T Cell Isolation Kit (Miltenyi Biotec) and cultured in Immunocult-XF T Cell Expansion Medium supplemented with 10 ng/mL IL-2 under different oxygen and pressure conditions. Cells were activated with anti-CD3/CD28 Human T-Activator Dynabeads. Cells were counted on days 3, 7, 10 and 4. FIG. 3A shows the growth curves for the T cells when grown under standard atmospheric conditions (20% O₂ at atmospheric pressure), 15% O₂ at atmospheric pressure, 15% O₂ at 2 PSI above atmospheric pressure, 5% O₂ at atmospheric pressure, 5% O₂ at 2 PSI above atmospheric pressure, 1% O₂ at atmospheric pressure, and, 1% O₂ at 2 PSI above atmospheric pressure. FIG. 3B shows a bar graph of the fold expansion of the T cells under the different conditions at day 14, and FIG. 3C shows the same data as a table. As seen in FIGS. 3A-3C, the cells cultured with 15% O₂ at 2 PSI above atmospheric pressure grew the fastest reaching almost 97 fold expansion compared to about 71-fold expansion for the T cells grown under standard conditions. At each different O₂ concentration, the cells cultured under hyperbaric conditions showed a greater fold expansion.

### Example 4: Effects of Hypoxic and Hyperbaric Culture Conditions on CD4 and CD8 Biomarker Expression

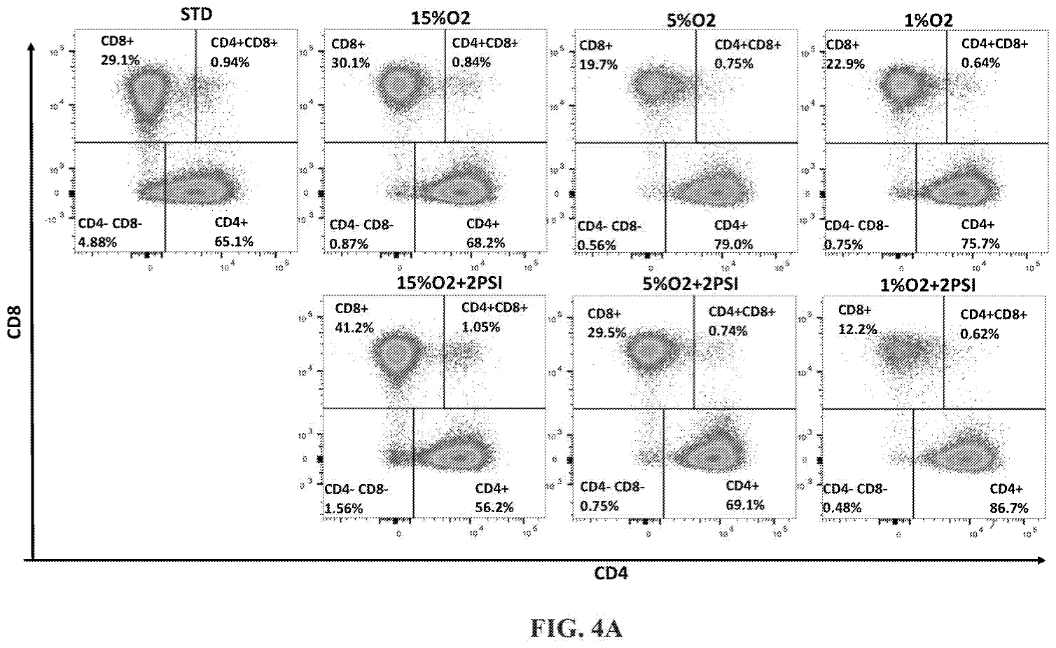

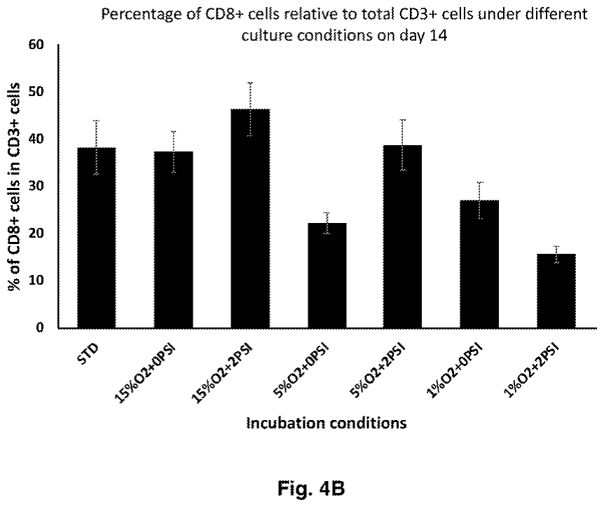

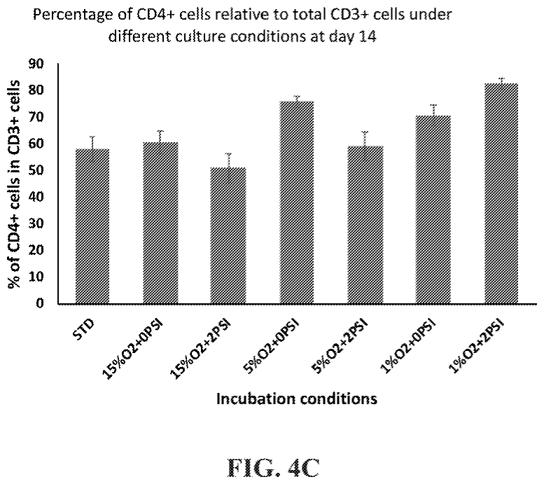

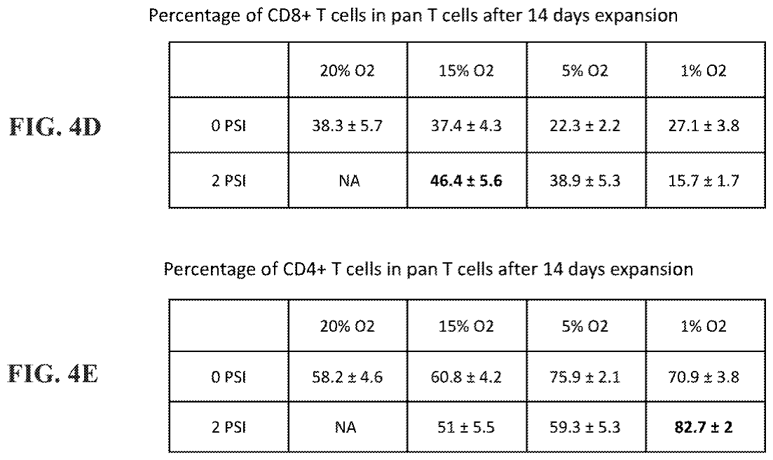

To assess the effects of hypoxic and hyperbaric culture conditions on T-cell phenotypes pan-T cells were cultured under different conditions for 14 days and assessed by fluorescent-assisted cell sorting (FACS) staining for CD4 and CD8. Cells were cultured under standard atmospheric conditions, 15% O₂ at atmospheric pressure, 15% O₂ at 2 PSI above atmospheric pressure, 5% O₂ at atmospheric pressure, 5% O₂ at 2 PSI above atmospheric pressure, 1% O₂ at atmospheric pressure, and, 1% O₂ at 2 PSI above atmospheric pressure. FIG. 4A shows representative plots of the cell sorting experiment, with CD8 expression shown on the vertical axis and CD4 expression shown on the horizontal axis. Percentages of cells which are CD4− CD8−, CD4+CD8−, CD4−CD8+, and CD4+CD8+ are shown as a percentage of the total number of pan-T cells analyzed (CD3+ cells). As shown in FIG. 4A, the percentage of CD8+ cells was increased after culture under 15% O₂ at 2 PSI above atmospheric pressure, but decreased after culture under 5% O₂ at atmospheric pressure, and under 1% O₂ at both pressures. The percentage of CD4+ cells was decreased by hyperbaric conditions at 15% and 5% O₂ conditions but increased by hyperbaric conditions at 1% O₂.

FIG. 4B shows the percentage of CD8+ cells relative to the total number of CD3+ pan-T cells; this data is also summarized in FIG. 4D. FIG. 4C shows the percentage of CD4+ cells relative to the total number of CD3+ pan-T cells; this data is also summarized in FIG. 4E. As shown in FIGS. 4A, 4B and 4D, the percentage of CD8+ cells was increased after culture under 15% O₂ at 2 PSI above atmospheric pressure, but decreased after culture under 5% O₂ at atmospheric pressure, and under 1% O₂ at both pressures. The percentage of CD4+ cells was decreased by hyperbaric conditions at 15% and 5% O₂ conditions but increased by hyperbaric conditions at 1% O₂, as seen in FIGS. 4A, 4C, and 4E.

### Example 5: Effects of Hypoxic and Hyperbaric Culture Conditions on Expression of Cytokines and Cytotoxicity Genes

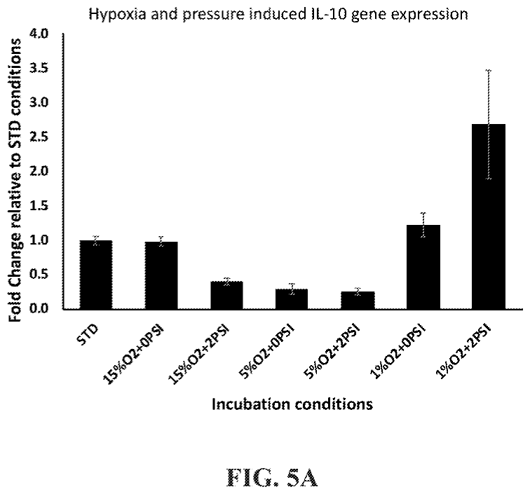

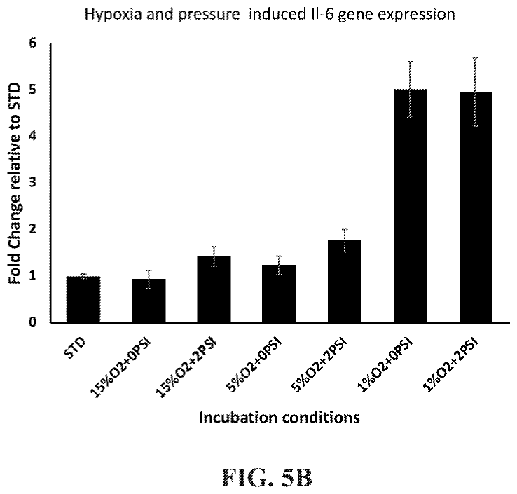

Pan-T cells were isolated from healthy donor PBMCs using a Human Pan-T Cell Isolation Kit (Miltenyi Biotec) and cultured in Immunocult-XF T cell expansion medium with IL-2 (10 ng/mL). Pan-T cells were activated using anti-CD3/CD28 Human T-Activator Dynabeads. The cells were cultured under different conditions for 3 days. Cells were collected on day 3, total RNA was isolated using Qiagen RNeasy plus Mini kit, and expression of cytokine and cytotoxic genes was analyzed by RT-qPCR. Expression of IL-10 was repressed under 15% O₂ and hyperbaric conditions, and at 5% O₂ under both atmospheric and hyperbaric conditions (FIG. 5A). Expression of IL-10 was upregulated under 1% O₂ conditions at both atmospheric and hyperbaric conditions (FIG. 5A). Expression of IL-6 was upregulated under the 1% O₂ hypoxic conditions at both atmospheric and hyperbaric conditions (FIG. 5B). Expression of IL-6 was not significantly increased at either 15% O₂ or 5% O₂ at atmospheric pressure but was increased at both O₂ levels under hyperbaric conditions (FIG. 5B).

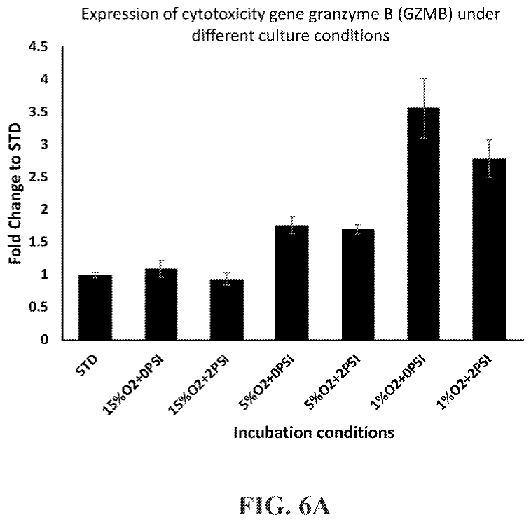

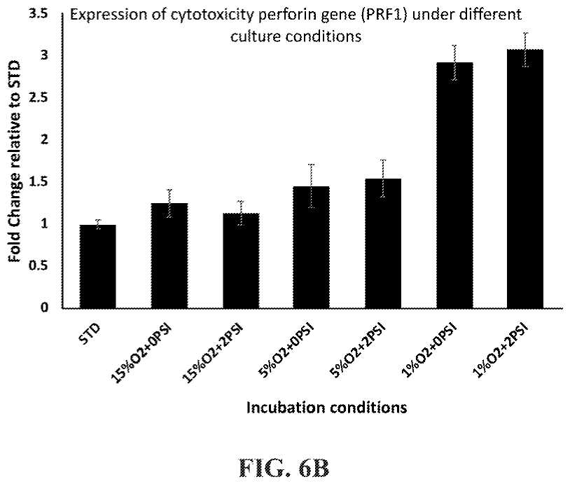

Expression of cytotoxicity genes granzyme B expression and perforin was upregulated by hypoxic conditions at 5% O₂ and 1% O₂, at both atmospheric and hyperbaric pressures, see FIGS. 6A and 6B.

### Example 6: Effects of Hypoxic and Hyperbaric Culture Conditions on Cell Size

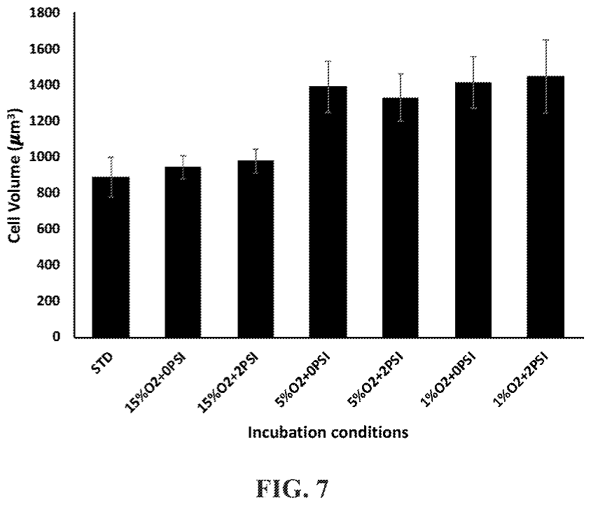

Pan-T cells were isolated from healthy donor PBMCs using a Human Pan-T Cell Isolation Kit (Miltenyi Biotec). Pan-T cells were cultured in Immunocult-XF medium with IL-2 (10 ng/mL) and activated using anti-CD3/CD28 Human T-Activator Dynabeads under hypoxic and hyperbaric culture conditions. Cell size was measured by Countess FL cell counter after 7 days of culture. Cell volumes of T cells that grew under hypoxic conditions at 5% O₂ and 1% O₂ were larger than cells cultured under standard O₂ levels or 15% O₂ levels (FIG. 7, n=6 unique donors).

### Example 7: Effects of Hypoxic and Hyperbaric Culture Conditions on Checkpoint Gene Expression

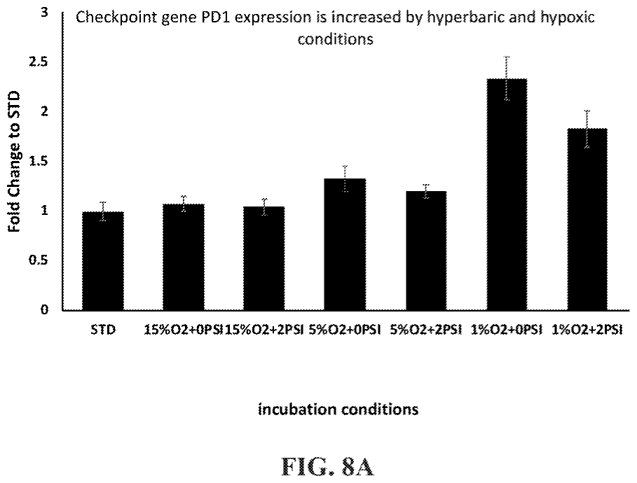

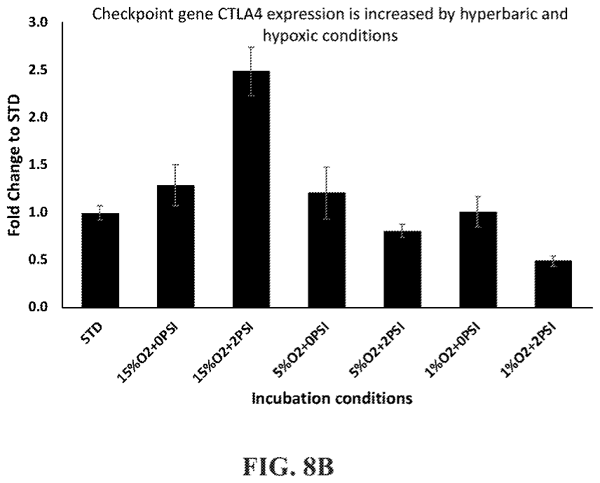

Pan-T cells from 3 unique donors were expanded in Immunocult-XF medium with 10 ng/mL IL-2 and activated using anti-CD3/CD28 Human T-Activator Dynabeads under different hypoxic and hyperbaric culture conditions. Cells were collected on day 3 for RNA isolation. Expression of checkpoint gene PD1 and CTLA4 was analyzed by RT-qPCR. As seen in FIG. 8A, expression of PD1 was increased at 5% O₂ and 1% O₂ at both atmospheric and hyperbaric pressures. Expression of CTLA4 was significantly upregulated at 15% O₂ and hyperbaric pressure and downregulated at 5% O₂ and 1% O₂ at hyperbaric pressure, as shown in FIG. 8B.

### Example 8: Effects of Hypoxic and Hyperbaric Culture Conditions on Cytokine Expression

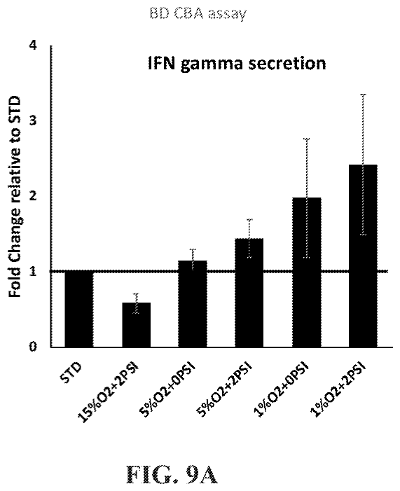

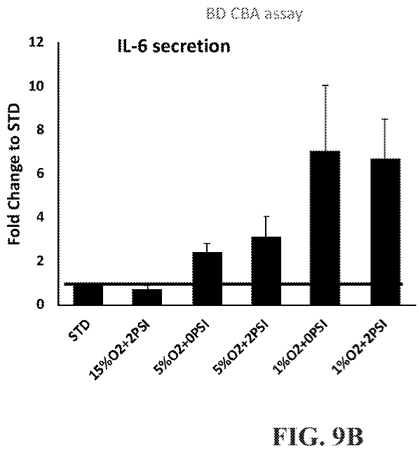

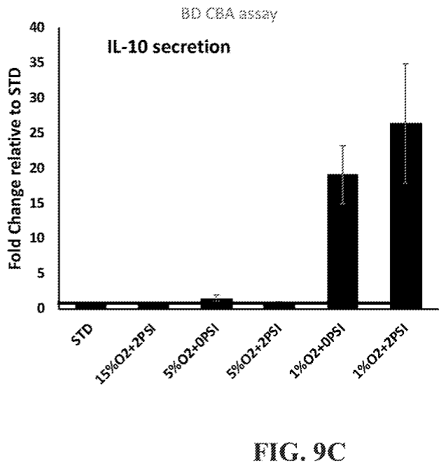

Pan-T cells were isolated and cultured under different hypoxic and hyperbaric conditions for 7 days. Supernatant was collected and secretion of IFN-gamma, IL-6, and IL-10 was assessed using a Cytometric Bead Array (CBA) Human Th1/Th2/Th17 Cytokine Kit (BD Biosciences). As seen in FIG. 9A expression of IFN-gamma was decreased under 15% O₂ and hyperbaric pressure, and was increased under 5% O₂ and hyperbaric pressure, and was increased under 1% O₂ at both atmospheric and hyperbaric pressures. Expression of IL-6 increased under 5% O₂ and 1% O₂ at both atmospheric and hyperbaric pressures, as seen in FIG. 9B. Expression of IL-10 was increased under 1% O₂ at both atmospheric and hyperbaric pressures (FIG. 9C).

### Example 9: Effects of Hypoxic and Hyperbaric Culture Conditions on Treg Cell Expression of FOXP3

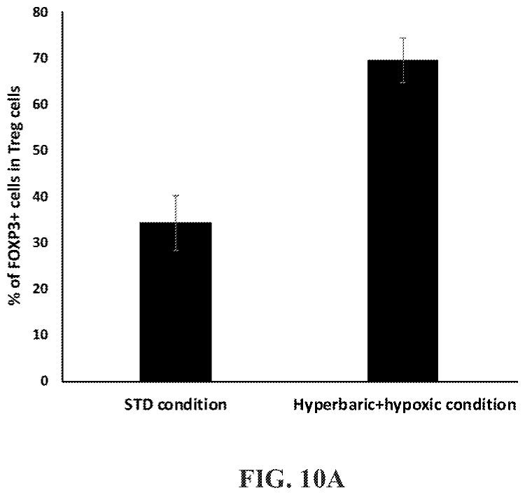

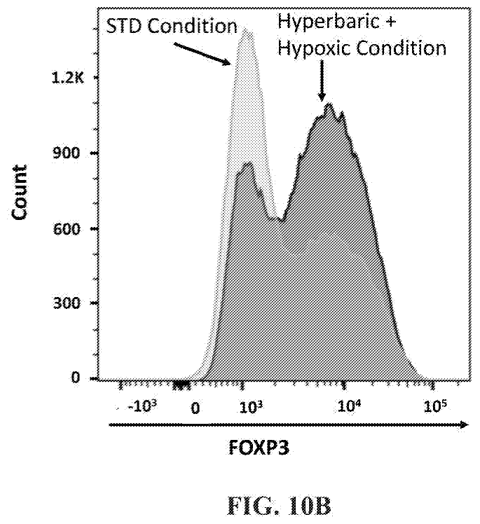

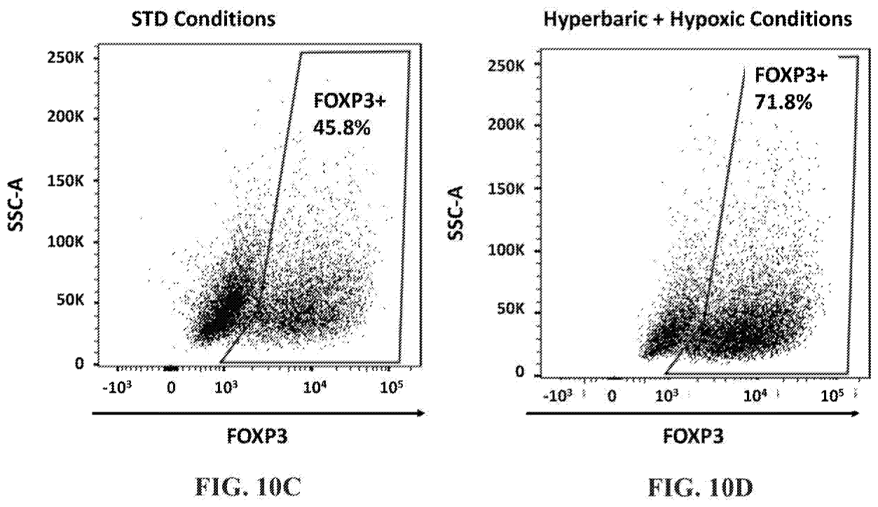

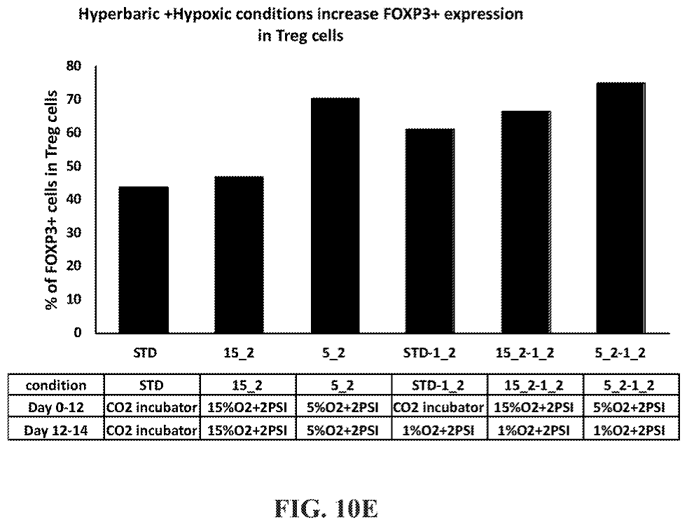

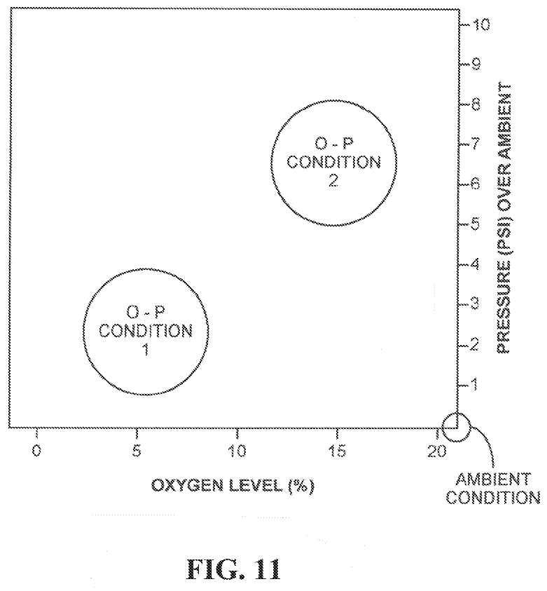

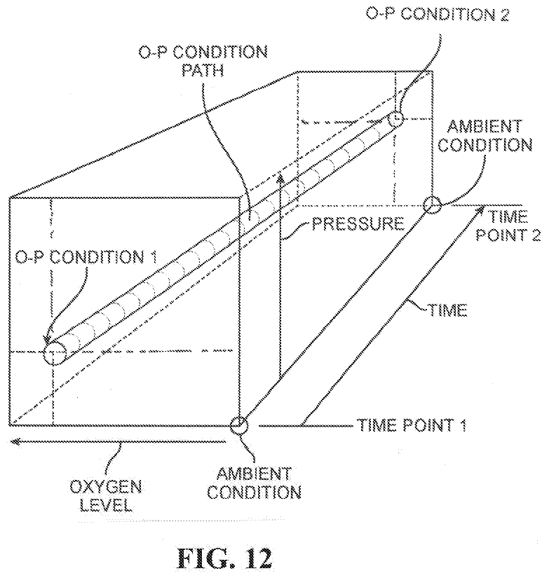

Treg cells were enriched by magnetic beads and cultured in under standard conditions (STD) or under hypoxic and hyperbaric conditions for 14 days. The percentage of FOXP3+ Treg cells was analyzed by flow cytometry. A greater percentage of Treg cells expressed FOXP3 when cultured under hypoxic and hyperbaric conditions (5% O₂ at 2 PSI above atmospheric pressure), as seen in FIGS. 10A and 10B. FIGS. 10C and 10D show representative scatter plats of cells grown under standard, and, hypoxic and hyperbaric conditions respectively.

To further assess the effects of hypoxic and hyperbaric culture conditions the Treg cells were enriched by magnetic beads and cultured under standard conditions and hypoxic and hyperbaric conditions (15% O₂ at 2 PSI above atmospheric pressure, and 5% O₂ at 2 PSI above atmospheric pressure) for 12 days. On Day 12, cells were equally split into two portions, one portion was cultured under the original conditions and the other portion was cultured under 1% O₂ at 2 PSI above atmospheric pressure for a further 2 days. On day 14, FOXP3+ positive Treg cells were analyzed by flow cytometry. As seen in FIG. 10E hypoxic and hyperbaric conditions produced more FOXP3+ Treg cells compared to standard conditions, and two-day culture under conditions of increased hypoxia further increased the proportion of FOXP3+ Treg cells compared original conditions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EMBODIMENTS

Embodiment 1. A method of modulating a phenotype of at least a subset of a source population of cells, the method including: culturing the source cell population in a liquid medium within a cell culture incubator that is configured to be able to regulate at least two variable atmospheric condition parameters within the incubator independently of a respective ambient atmospheric condition, wherein two of the variable atmospheric parameters are an oxygen level and a total atmospheric pressure level; regulating at least one of the oxygen level and the total atmospheric pressure level within the incubator such that at least one of the oxygen level or the total atmospheric pressure level differs from the respective ambient level; and as a consequence of the regulating of the variable atmospheric condition parameters, driving expression of a phenotypic parameter of the source population, over an incubation period, from a first phenotype toward a second phenotype, wherein the first phenotype of the subset cell population is that which would be expressed under an atmospheric condition in which the variable atmospheric condition parameters within the incubator were substantially the same as ambient atmospheric conditions, and wherein the second phenotype of the subset cell population is expressed as a consequence of exposure to the variable atmospheric conditions, as regulated by the incubator.

Embodiment 2. The method of embodiment 1, wherein the first and second phenotypes comprise indicators of one or more observable parameters of cell culture performance in vitro.

Embodiment 3. The method of any one of embodiments 1-2 wherein the one or more parameters of cell culture parameters are selected from the group consisting of growth rate, cell death rate, achievable cell density, rate of production of a cell product (natural or transfection-based), cell morphology, cell dimension, cell adherent properties, cell electrical properties, cell metabolic activity, cell migratory behavior, cell activation state, cell differentiation state, biomarker demonstration, amenability or resistance to transfection, vulnerability or resistance to infection, amenability or resistance to viral transduction, responsiveness or resistance to a bioactive agent, or any other observable aspect of cell phenotype or function.

Embodiment 4. The method of any one of embodiments 1-3, wherein the second phenotype is suitable for human therapeutic application, said application being selectable from the group consisting of patient treatment, drug screening in a drug development assay, and testing a patient-specific sensitivity to a candidate drug.

Embodiment 5. The method of any one of embodiments 1-4 wherein the oxygen level within the incubator is regulated to a hypoxic level with respect to the ambient oxygen level.

Embodiment 6. The method of any one of embodiments 1-5 wherein the total atmospheric pressure is regulated to a hyperbaric level with respect to the ambient atmospheric pressure.

Embodiment 7. The method of any one of embodiments 1-6 wherein the oxygen level is regulated to a hypoxic level with respect to the ambient oxygen level, and wherein the total atmospheric pressure is regulated to a hyperbaric level with respect to the ambient atmospheric pressure.

Embodiment 8. The method of any one of embodiments 1-7 wherein the total atmospheric pressure is regulated to a hyperbaric level and wherein the oxygen level is regulated to a hypoxic level, the atmospheric pressure and the oxygen level being regulated independently of each such that a hypoxic oxygen level prevails in spite of an overall hyperbaric condition.

Embodiment 9. The method of any one of embodiments 1-8 wherein the level of oxygen is in the range of about 0.1% to about 20%.

Embodiment 10. The method of any one of embodiments 1-9 wherein the level of oxygen is in the range of about 1% to about 15%.

Embodiment 11. The method of any one of embodiments 1-10 wherein the level of oxygen is in the range of about 2% to about 10%.

Embodiment 12. The method of any one of embodiments 1-11 wherein the total atmospheric gas pressure is greater than that of an ambient atmospheric pressure by a value in the range of about 0.1 PSI to about 10 PSI.

Embodiment 13. The method of any one of embodiments 1-12 wherein the total atmospheric gas pressure is greater than that of an ambient atmospheric pressure by a value in the range of about 1 PSI to about 6 PSI.

Embodiment 14. The method of any one of embodiments 1-13 wherein the total atmospheric gas pressure is greater than that of an ambient atmospheric pressure by a value in the range of about 2 PSI to about 5 PSI.

Embodiment 15. The method of any one of embodiments 1-14 wherein a third variable parameter of the atmospheric conditions includes a carbon dioxide level or a temperature.

Embodiment 16. The method of embodiment 15 wherein the temperature ranges between about 33° C. and about 40° C.

Embodiment 17. The method of embodiment 15 wherein the level of carbon dioxide in the atmosphere exerts an effect on a pH of the liquid medium, and wherein pH comprises a further variable parameter capable of contributing to driving the subset population to the second phenotype.

Embodiment 18. The method of any one of embodiments 1-17 wherein the subset population of the first phenotype includes a phenotypic plasticity that is responsive to a combination of the two or more variable atmospheric parameters, said plasticity supporting the driving of the first phenotype toward the second phenotype.

Embodiment 19. The method of any one of embodiments 1-18 wherein driving the subset population from a first phenotype to a second phenotype occurs in the absence of a coinciding transfecting method.

Embodiment 20. The method of any one of embodiments 1-18 wherein driving the subset population from a first phenotype to a second phenotype occurs in conjunction with a coinciding transfecting method.

Embodiment 21. The method of any one of embodiments 1-20 wherein the relative growth rate of the second phenotype may be any of greater than that of the first phenotype, substantially equivalent to that of the second phenotype, or less than that of the first phenotype.

Embodiment 22. The method of any one of embodiments 1-21 wherein modulating a phenotype of at least a subset population of the source population includes changing a relative presence of the subset population within the source population.

Embodiment 23. The method of embodiment 22 wherein changing a relative presence of the subset population within the source population includes an increase in a net growth rate of the subset population in contrast to other subset populations within the source cell population.

Embodiment 24. The method of embodiment 22 wherein changing a relative presence of the subset population within the source population includes a switching of an individual cell from a first phenotype to a second phenotype.

Embodiment 25. The method of any one of embodiments 1-24 wherein modulating a phenotype of at least a subset population of the source population includes driving a change in phenotype that would not occur absent the regulating of the two or more variables of atmospheric condition within the incubator.

Embodiment 26. The method of any one of embodiments 1-24 wherein modulating a phenotype of at least a subset population of the source population includes accelerating a change in phenotype that could occur absent the regulating of the two or more variables of atmospheric condition within the incubator.

Embodiment 27. The method of any one of embodiments 1-26 wherein the two or more variable parameters of the atmospheric conditions comprise an oxygen level and a total gas pressure, culturing the cells within a cell culture incubator includes culturing for a culture run over which time both the oxygen level and total gas pressure are substantially constant.

Embodiment 28. The method of any one of embodiments 1-27 wherein the two or more variable parameters of the atmospheric conditions comprise an oxygen level and a total gas pressure, and culturing the cells within a cell culture incubator includes varying at least one of the oxygen level or the total gas pressure over a culture run.

Embodiment 29. The method of embodiment 28 wherein varying at least one of the total gas pressure and the at least one individual gas during the culture duration includes any of increasing or decreasing any one or more of the total gas pressure and the concentration of the at least one individual gas during the culture duration.

Embodiment 30. The method of embodiment 28 wherein varying any one or more of the total gas pressure and the concentration of the at least one individual gas includes varying as a ramping function.

Embodiment 31. The method of embodiment 28 wherein varying any one or more of the total gas pressure and the concentration of the at least one individual gas includes varying as a step function.

Embodiment 32. The method of embodiment 31 wherein the step function occurs over an elapsed time that is sufficient for dissolved gas levels in a liquid cell culture medium to come into equilibrium with atmospheric conditions to which the medium is exposed.

Embodiment 33. The method of embodiment 28 wherein varying any one or more of the total gas pressure and the concentration of the at least one individual gas includes culturing under a first set of gaseous conditions and culturing under at least a second set of gaseous condition.

Embodiment 34. The method of embodiment 33 wherein culturing under a first gaseous condition and culturing under at least a second gaseous condition includes moving from the first condition to the at least second condition and back to the first condition one or more times.

Embodiment 35. The method of embodiment 28 wherein varying any one or more of the total gas pressure and the at least one individual gas includes synchronously changing (a) the total gas pressure and (b) the concentration of the at least one gas.

Embodiment 36. The method of embodiment 28 wherein varying any one or more of the total gas pressure and the at least one individual gas includes asynchronously changing (a) the total gas pressure and (b) the concentration of the at least one gas.

Embodiment 37. The method of any one of embodiments 1-36, wherein the liquid medium includes one or more bioactive agents.

Embodiment 38. The method of embodiment 37 wherein the bioactive agent includes any of a nucleic acid, a peptide, a protein, or a lipid.

Embodiment 39. The method of embodiment 37 wherein the bioactive agent includes any of a transcription factor, a cytokine, or a growth factor.

Embodiment 40. The method of any one of embodiments 1-39, wherein regulating the two or more variable parameters of the atmospheric condition includes establishing or approaching an equilibrium between one or more individual gases in a gas phase head space and the one or more gases dissolved in the liquid medium, wherein the equilibrium is established at a direct gas-liquid interface.

Embodiment 41. The method of any one of embodiments 1-40 wherein the source population includes cell populations selected from the group consisting of an immune cell population, a tumor cell population, and a stem cell population.

Embodiment 42. The method of embodiment 41 wherein the source population is an immune cell population, and wherein a phenotypic shift from a first phenotype to a second phenotype is modulated by exposure to hypoxic atmospheric condition, wherein the hypoxic condition includes an oxygen level range between about 1% and about 15%.

Embodiment 43. The method of embodiment 41 wherein the source population is an immune cell population, and wherein a phenotypic shift from a first phenotype to a second phenotype is modulated by exposure to hyperbaric atmospheric condition, wherein the hyperbaric condition includes an atmospheric pressure range of about 2 PSI over the ambient atmospheric pressure level.

Embodiment 44. The method of embodiment 41 wherein the cell populations comprise cell populations derived that are not genetically engineered.

Embodiment 45. The method of embodiment 41 wherein the cell populations comprise cell populations derived that have been genetically engineered.

Embodiment 46. The method of embodiment 41 wherein the cell populations comprise cell populations derived from any of healthy donor individuals or patients that have an illness pertinent to the source cell population, or a history of thereof.

Embodiment 47. The method of embodiment 41 wherein the source population comprises a lymphocyte population from a human donor.

Embodiment 48. The method of embodiment 41 wherein the immune cell population comprises a human immune cell population.

Embodiment 49. The method of embodiment 48 wherein driving expression of a phenotypic parameter comprises immunomodulating an immune cell's immunological functionality within an immune system.

Embodiment 50. The method of embodiment 49 wherein immunomodulating an immune cell functionality comprises an activation of the immune cell functionality.

Embodiment 51. The method of embodiment 49 wherein immunomodulating an immune cell functionality comprises a suppression of the immune cell functionality.

Embodiment 52. The method of embodiment 41 wherein the source population of immune cells comprises a peripheral blood mononuclear cell (PBMC) population from a human donor.

Embodiment 53. The method of embodiment 52 wherein the variable atmospheric parameters that drive the PBMC population toward the second phenotype include an oxygen level of about 15% and a total atmospheric pressure of about 0PST to about 2 PSI over an ambient level.

Embodiment 54. The method of embodiment 52 wherein the second phenotype of the PBMC population shows a decreased level of IL-10 secretion compared to that of the first phenotype.

Embodiment 55. The method of embodiment 54 wherein the variable atmospheric parameters that drive the PBMC population toward the second phenotype include an oxygen level of about 15% and a total atmospheric pressure of about 2 PSI over an ambient level.

Embodiment 56. The method of embodiment 52 wherein the second phenotype of the PBMC population shows an increased level of TNFα secretion as compared to that of the first phenotype.

Embodiment 57. The method of embodiment 56 wherein the variable atmospheric parameters that drive the PBMC population toward the second phenotype include an oxygen level of about 15% and a total atmospheric pressure of about 0 PSI over an ambient level.

Embodiment 58. The method of embodiment 52 wherein the second phenotype of the PBMC population shows an increased ability to kill prostate cancer cells with Novolumab, as compared to that of the first phenotype.

Embodiment 59. The method of embodiment 58 wherein the variable atmospheric parameters that drive the PBMC population toward the second phenotype include an oxygen level of about 1% and a total atmospheric pressure of about 2 PSI over an ambient level.

Embodiment 60. The method of embodiment 52 wherein the second phenotype of the PBMC population shows an increased ability to kill prostate cancer cells with Pembro, as compared to that of the first phenotype.

Embodiment 61. The method of embodiment 60 wherein the variable atmospheric parameters that drive the PBMC population toward the second phenotype include an oxygen level of about 1% and a total atmospheric pressure of about 2 PSI over an ambient level.

Embodiment 62. The method of embodiment 41 wherein the source population of immune cells comprises a Pan-T cell population from a human donor.

Embodiment 63. The method of embodiment 62 wherein the variable atmospheric parameters that drive the Pan-T cell population toward the second phenotype include an oxygen level of about 1% to about 15% and a total atmospheric pressure of about 0 PSI to about 2 PSI over an ambient level.

Embodiment 64. The method of embodiment 62 wherein the second phenotype of the Pan-T cell population shows an increased growth rate as compared to that of the first phenotype.

Embodiment 65. The method of embodiment 64 wherein the variable atmospheric parameters that drive the Pan-T cell population toward the second phenotype include an oxygen level of about 15% and a total atmospheric pressure of about 2 PSI over an ambient level.

Embodiment 66. The method of embodiment 62 wherein the second phenotype of the Pan-T cell population shows an increased final cell density as compared to that of the first phenotype.

Embodiment 67. The method of embodiment 66 wherein the variable atmospheric parameters that drive the Pan-T cell population toward the second phenotype include an oxygen level of about 15% and a total atmospheric pressure of about 2 PSI over an ambient level.

Embodiment 68. The method of embodiment 62 wherein the second phenotype of the Pan-T cell population shows a shift in relative presence of CD8+ cells to CD4+ cells as compared to those of the first phenotype.

Embodiment 69. The method of embodiment 68 wherein the variable atmospheric parameters that drive the Pan-T cell population toward the second phenotype include an oxygen level of about 1% and a total atmospheric pressure of about 2 PSI over an ambient level.

Embodiment 70. The method of embodiment 62 wherein the second phenotype of the Pan-T cell population shows and increased expression of cytokine IL-6 as compared to that of the first phenotype.

Embodiment 71. The method of embodiment 70 wherein the variable atmospheric parameters that drive the Pan-T cell population toward the second phenotype include an oxygen level of about 1% and a total atmospheric pressure of about 2 PSI over an ambient level.

Embodiment 72. The method of embodiment 62 wherein the second phenotype of the Pan-T cell population shows an increased expression of cytokine IL-10 as compared to that of the first phenotype.

Embodiment 73. The method of embodiment 72 wherein the variable atmospheric parameters that drive the Pan-T cell population toward the second phenotype include an oxygen level of about 1% and a total atmospheric pressure of about 2 PSI over an ambient level.

Embodiment 74. The method of embodiment 62 wherein the second phenotype of the Pan-T cell population shows an increased expression of cytotoxicity gene GZMB expression is increased as compared to that of the first phenotype.

Embodiment 75. The method of embodiment 75 wherein the variable atmospheric parameters that drive the Pan-T cell population toward the second phenotype include an oxygen level of about 1% and a total atmospheric pressure of about 2 PSI over an ambient level.

Embodiment 76. The method of embodiment 62 wherein the second phenotype of the Pan-T cell population shows an increased expression cytotoxicity gene perforin as compared to that of the first phenotype.

Embodiment 77. The method of embodiment 76 wherein the variable atmospheric parameters that drive the Pan-T cell population toward the second phenotype include an oxygen level of about 1% and a total atmospheric pressure of about 2 PSI over an ambient level.

Embodiment 78. The method of embodiment 62 wherein the second phenotype of the Pan-T cell population shows an increase in cell volume is increased as compared to that of the first phenotype.

Embodiment 79. The method of embodiment 78 wherein the variable atmospheric parameters that drive the Pan-T cell population toward the second phenotype include an oxygen level of about 1% and a total atmospheric pressure of about 2 PSI over an ambient level.

Embodiment 80. The method of embodiment 62 wherein the second phenotype of the Pan-T cell population shows an increase in the expression of checkpoint gene PD1 as compared to that of the first phenotype.

Embodiment 81. The method of embodiment 80 wherein the variable atmospheric parameters that drive the Pan-T cell population toward the second phenotype include an oxygen level of about 1% and a total atmospheric pressure of about 0 PSI over an ambient level.

Embodiment 82. The method of embodiment 62 wherein the second phenotype of the Pan-T cell population shows an increased in the expression of checkpoint gene CTLA4 as compared to that of the first phenotype.

Embodiment 83. The method of embodiment 82 wherein the variable atmospheric parameters that drive the Pan-T cell population toward the second phenotype include an oxygen level of about 15% and a total atmospheric pressure of about 2 PSI over an ambient level.

Embodiment 84. The method of embodiment 62 wherein the second phenotype of the Pan-T cell population shows an increased level of IFN gamma secretion as compared to that of the first phenotype.

Embodiment 85. The method of embodiment 84 wherein the variable atmospheric parameters that drive the Pan-T cell population toward the second phenotype include an oxygen level of about 1% and a total atmospheric pressure of about 2 PSI over an ambient level.

Embodiment 86. The method of embodiment 62 wherein the second phenotype of the Pan-T cell population shows an increased level of IL-6 secretion as compared to that of the first phenotype.

Embodiment 87. The method of embodiment 86 wherein the variable atmospheric parameters that drive the Pan-T cell population toward the second phenotype include an oxygen level of about 1% and a total atmospheric pressure of about 2 PSI over an ambient level.

Embodiment 88. The method of any embodiment 41 wherein the source population of immune cells comprises a Treg cell population from a human donor.

Embodiment 89. The method of any embodiment 88 wherein the second phenotype of the Treg cell population shows an increased rate of expression of FOXP3 as compared to that of the first phenotype.

Embodiment 90. The method of embodiment 89 wherein the variable atmospheric parameters that drive the Treg cell population toward the second phenotype include an oxygen level of about between about 1% to about 5% and a total atmospheric pressure of about 2 PSI over an ambient level.

Embodiment 91. The method of embodiment 41 wherein the immune cell populations comprise hematopoietic stem cell and descendant lineage populations.

Embodiment 92. The method of embodiment 91 wherein the descendant immune cell lineage populations are selected from the group consisting of populations of common lymphoid progenitor cells and populations of common myeloid progenitor cells.

Embodiment 93. The method of embodiment 92 wherein the common lymphoid progenitor cell populations are selected from the group consisting of B cell, natural killer (NK) cell, T cell, and dendritic cell populations.

Embodiment 94. The method of embodiment 92 wherein the common myeloid progenitor cell populations are selected from the group consisting of granulocyte macrophage progenitor cell, and megakaryocyte erythroid progenitor cell populations.

Embodiment 95. The method of embodiment 94 wherein the granulocyte macrophage progenitor cell populations are selected from the group consisting of monocyte and myeloblast populations.

Embodiment 96. The method of embodiment 95 wherein the monocyte populations are selected from the group consisting of monocyte-derived dendritic cells and macrophages.

Embodiment 97. The method of embodiment 95 wherein the myeloblast populations are selected from the group consisting of neutrophil, eosinophil, and basophil populations.

Embodiment 98. The method of embodiment 94 wherein the megakaryocyte erythroid progenitor cell populations are selected from the group consisting of erythrocyte and megakaryocyte populations.

Embodiment 99. The method of embodiment 41 wherein the tumor cell populations are sourced from cancers selected from the group consisting of ovarian cancer, acute myeloid leukemia, pancreatic cancer, non-small cell lung carcinoma (NSCLC), colorectal tumor, prostate cancer, hepatic cancer, mesothelioma, and melanoma.

Embodiment 100. The method of embodiment 41 wherein the stem cell populations comprise mesenchymal stem cells, said stem cells including progenitor, immature and mature subgroups.

Embodiment 101. The method of any one of embodiments 1-100, wherein the cell population of the second phenotype is a targeted product of the method, and wherein regulating the two or more variable parameters of the atmospheric condition includes selecting values for the two or more variable parameters that favor expression of the targeted product.

Embodiment 102. The method of embodiment 101, wherein the selection of conditions is based on experimental data from previous examples of a population of cells of that is a same or a similar type of cell population as the source population being used in an implementation of the method.

Embodiment 103. The method of embodiment 101 wherein the second phenotype is desirable because of its manifesting any one or more cell culture performance parameters, said parameters selected from the group consisting of cell growth rate, cell death rate, achievable cell density, rate of production of a cell product (natural or transfection-based), cell morphology, cell dimension, cell adherent properties, cell electrical properties, cell metabolic activity, cell migratory behavior, cell activation state, cell differentiation state, biomarker demonstration, amenability or resistance to transfection, vulnerability or resistance to infection, amenability or resistance to viral transduction, responsiveness or resistance to a bioactive agent, or any other observable aspect of cell phenotype or function.

Embodiment 104. The method of embodiment 103 wherein cell activation state comprises an immune cell activation state, and wherein driving expression of a phenotypic parameter comprises any of a driving toward a relatively high immunological cell functioning state or driving to a relatively suppressed immunological cell functioning state.

Embodiment 105. The method of any one of embodiments 1-104, further including expanding the population of cells of the second phenotype by way of culturing them further.

Embodiment 106. The method of any one of embodiments 1-105, wherein a particular second phenotype is desired, and wherein a set of the two or more independently regulated parameters of the atmospheric conditions that favor an expression of the desired second phenotype has been determined, the method further includes expanding the cultured subset of cells of expressing the second phenotype by a culturing cells of the second phenotype under those two or more independently regulated atmospheric conditions.

Embodiment 107. The method of embodiment 106 further including determining values for the two or more independently regulated parameters of the atmospheric conditions that favor the expression of the desired second phenotype, the method including: splitting the source population of cells into cohort cultures including at least a first and a second cohort culture; measuring a cell culture performance parameter indicative of the desired phenotype within each of the cohort cultures; and based on the results of the cell culture performance parameter among the cohort cultures, determining which of the variations in atmospheric conditions is optimal for the outgrowth of the cell population having the desired phenotype.

Embodiment 108. The method of any one of embodiments 1-107, wherein the subset cell population of the second phenotype is directed toward further culturing in a drug or drug candidate testing format.

Embodiment 109. A method of increasing phenotypic homogeneity of a phenotype of a cell population derived from an initial source population of cells, the method including: culturing the source cell population in a liquid medium within a cell culture incubator configured to regulate two or more variable parameters of an atmospheric condition within the incubator independently of any respective ambient atmospheric condition, wherein the phenotype of the source population initially includes an initial level of variability with respect to one or more parameters of cell culture performance; regulating the two or more variable parameters of the atmospheric condition within the incubator such that at least one of the variable parameters differs from the ambient level of the respective variable parameter; and as a consequence of culturing the source cell population under the regulated atmospheric condition, diminishing the level of variability with respect to the one or more parameters of cell culture performance, thus yielding a later population of cells with a level of phenotypic homogeneity greater than that of the source population of cells.

Embodiment 110. The method of embodiment 109 further including applying the later population of cells as a substrate for testing efficacy of drugs or drug candidates.

Embodiment 111. The method of embodiment 110, wherein the drugs or drug candidates are considered to be possibly cytotoxic to the later population of cells.

Embodiment 112. The method of embodiment 1110, wherein the drugs or drug candidates are considered to be possibly supportive of the later population of cells.

Embodiment 113. A method of stabilizing a phenotype of at least a subset population of a source population of cells, the method including: culturing the source cell population in a liquid medium within a cell culture incubator that is configured to regulate atmospheric parameters within the incubator, wherein the atmospheric parameters comprise an oxygen level and a total gas pressure level; regulating the atmospheric parameters within the incubator such that at least one of the atmospheric parameters differs from an ambient level thereof; and as a consequence of regulating the atmospheric parameters, stabilizing the cell population as a first phenotype, wherein a second phenotype is that toward which the cell population would drift under an atmospheric condition in which the variable atmospheric parameters within the incubator were substantially in accord with ambient conditions.

Embodiment 114. The method of embodiment 113 wherein the source population includes cell populations selected from the group consisting of immune cells, tumor cells, and stem cells.

Embodiment 115. The method of embodiment 113 wherein the first phenotype continues to be expressed as a consequence of exposure to the atmospheric conditions as regulated within the incubator for a duration of a cell culture run, and wherein the second phenotype would be expressed if variable atmospheric parameters within the incubator were substantially in accordance with ambient conditions.

Embodiment 116. A method of determining values for an oxygen level value and a total gas pressure value in an atmosphere overlaying a liquid cell culture medium that collectively favor an expression of a desired phenotype of a source population of cells being cultured in the medium, the method including: splitting the source population of cells into cohort cultures including at least a first and a second cohort culture; culturing the cohort cell cultures in parallel under atmospheric conditions that differ only with regard for variations in any of oxygen concentration and total gas pressure; measuring a cell culture performance parameter indicative of the desired phenotype within each of the cohort cultures; and based on the results of the cell culture performance parameter among the cohort cultures, determining which oxygen and which total gas pressure levels are optimal for the outgrowth of the cell population having the desired phenotype.

Embodiment 117. The method of embodiment 116 further including, prior to initiating the method, determining a character of the desired phenotype as reflected in one or more parameters of cell culture performance.

Embodiment 118. The method of embodiment 116, wherein the cell culture performance parameters are selected from the group consisting of growth rate, cell death rate, achievable cell density, rate of production of a cell product (natural or transfection-based), cell morphology, cell dimension, cell adherent properties, cell electrical properties, cell metabolic activity, cell migratory behavior, cell activation state, cell differentiation state, biomarker demonstration, amenability or resistance to transfection, vulnerability or resistance to infection, amenability or resistance to viral transduction, responsiveness or resistance to a bioactive agent, or any other observable aspect of cell phenotype or function.

Embodiment 119. The method of embodiment 116 wherein the source population includes cell populations selected from the group consisting of immune cells, tumor cells, and stem cells.

Embodiment 120. A method of determining levels for an oxygen level and a total gas pressure within a cell culture incubator that favor expression of an immune cell population phenotype that is optimally responsive to the presence of a bioactive agent, the method comprising: splitting the immune cell population into multiple cohort cell cultures; culturing the cohort cell cultures in parallel under atmospheric conditions that differ only with regard to variations in any of oxygen level or total gas pressure; measuring a cell culture performance parameter that is responsive to the immune cell-directed bioactive agent in each cohort culture; and based on the measurement of the cell culture performance parameter among the cohort cultures, determining which of the oxygen and total gas pressure levels support a maximal responsiveness among the cohort immune cell cultures to the bioactive agent.

Embodiment 121. The method of embodiment 120 wherein the cell culture performance parameters comprise any one or more of growth rate, cell death rate, achievable cell density, rate of production of a cell product (natural or transfection-based), cell morphology, cell dimension, cell adherent properties, cell electrical properties, cell metabolic activity, cell migratory behavior, cell activation state, cell differentiation state, biomarker demonstration, amenability or resistance to transfection, vulnerability or resistance to infection, amenability or resistance to viral transduction, responsiveness or resistance to a bioactive agent, or any other observable aspect of cell phenotype or function.

Embodiment 122. The method of embodiment 121 wherein an effect of the bioactive agent increases the magnitude of the cell culture performance parameter response.

Embodiment 123. The method of embodiment 121 wherein an effect of the bioactive agent decreases the magnitude of the cell culture performance parameter response.

Embodiment 124. A method of testing efficacy of an anti-cancer agent on patient-derived cancer cell including: expanding a cell population derived from a patient's tumor in a liquid medium under overlaying atmospheric conditions known or presumed to be supportive of growing cells from tumors like that of the patient, wherein atmospheric conditions comprise a hypoxic level of oxygen and a hyperbaric level of total gas pressure, and wherein expanding the cell population includes expanding to a level sufficient to seed multiple cohort cultures; splitting the expanded cell population into multiple cohort cell cultures; culturing the cohort cell cultures in parallel under conditions that are identical except for presence of one or more anti-cancer agents and under atmospheric conditions known or presumed to be supportive of expressing a cell phenotype that is optimal for testing efficacy of an anti-cancer agent, wherein the atmospheric conditions comprise a hypoxic level of oxygen and a hyperbaric level of total gas pressure; measuring a cell culture performance parameter that is affected by the anti-cancer agent in each cohort culture; and based on the measurement of the cell culture performance parameter among the cohort cultures, predicting efficacy of the one or more anti-cancer agents in treating the patient's tumor.

Embodiment 125. The method of embodiment 124, wherein the anticancer agent is comprised within a formulation for clinical use.

Embodiment 126. The method of embodiment 125, wherein the formulation includes one or more further anti-cancer agents.

Embodiment 127. A method of modulating phenotypic expression of a cell culture population to achieve a targeted phenotype, the method including: incubating a source cell population in a cell culture incubator configured to operate an atmospheric condition-controlling incubator program, wherein said program directs atmospheric conditions that optimize expression of a targeted phenotype, said program comprising set point ranges for (1) an oxygen level and (2) a total gas pressure level; regulating oxygen level and total gas pressure within the incubator in accordance with the atmospheric condition set point ranges; and culturing the cell population in accordance with said atmospheric condition set point ranges for sufficient culture duration to yield an expanded population of cells that express the targeted phenotype, wherein the expanded cell population comprises potential use as a human therapeutic.

Embodiment 128. The method of embodiment 127 wherein the expanded cell population further includes potential use in any one or more applications in the group consisting of a research model, a drug-screening model for use in drug or candidate drug development, or a patient-specific model for testing the efficacy of a drug or candidate drug.

Embodiment 129. The method of embodiment 127 wherein, prior to regulating oxygen level and total gas pressure within the incubator in accordance with the atmospheric condition modules that favor the targeted phenotype, the method further includes experimentally determining the oxygen level and total gas pressure conditions that favor the targeted phenotype.

Embodiment 130. The method of embodiment 129 wherein the oxygen level and total gas pressure conditions that favor the targeted phenotype are based on any of experimental data from previous examples of a population of cells of a same type as the source population or data from previous examples of populations of cells similar to those of the source population.

Embodiment 131. The method of embodiment 127 wherein culturing a source cell population includes culturing under clinical manufacturing conditions.

Embodiment 132. The method of embodiment 127 further including packaging the expanded population of cells expressing the targeted phenotype in a packaging that is appropriate for clinical use.

Embodiment 133. A method of modulating phenotypic expression of a cell culture population to achieve an optimal manufacturing process efficiency, the method including: culturing a source cell population in a liquid medium within a cell culture incubator configured to operate a first and a second atmospheric condition-controlling module, wherein each module includes set point ranges for an oxygen level and a total gas pressure level, wherein the first and second atmospheric condition modules are different from each other; and regulating the oxygen level and the total gas pressure within the incubator in a first phase and a second phase over the course of a cell culture run, wherein the first phase is operated according to atmospheric condition-controlling module that is optimized to support expansion of a population of cells that includes potential to express the targeted phenotype, and wherein the second phase is operated according to the second atmospheric condition-controlling module that is optimized to support expression of the targeted phenotype.

Embodiment 134. The method of modulating the phenotypic expression of a cell culture population according to embodiment 133, wherein the set point range for the oxygen level in the first phase of the cell culture run is higher than the oxygen level in the second phase of the cell culture run.

Embodiment 135. The method of modulating the phenotypic expression of a cell culture population according to embodiment 133, wherein a product of the method includes the cell population, said population including a potential use as a human therapeutic.

Embodiment 136. The method of modulating the phenotypic expression of a cell culture population according to embodiment 133, wherein a product of the method includes a cell-based product, the product including a potential use as a human therapeutic.

Embodiment 137. A product made by way of modulating a phenotype of at least a subset population of a source population of cells, the method including: culturing the source cell population in a cell culture incubator that is able regulate at least two variable parameters of an atmospheric condition within the incubator independently of any respective ambient atmospheric condition, wherein said parameters comprise an oxygen level and a total gas pressure level; regulating the variable parameters of the atmospheric condition within the incubator such that at least one of them differs from the ambient level of the respective variable parameter; and as a consequence of the regulating the atmospheric condition, driving the subset population from a first phenotype toward a second phenotype wherein the first phenotype of the subset cell population is that which would be expressed under an atmospheric condition in which the variable atmospheric parameters within the incubator were substantially the same as ambient conditions, and wherein the second phenotype of the subset cell population is expressed as a consequence of exposure to the atmospheric conditions, as regulated within the incubator; and collecting the product, wherein the product is a cell population of the second phenotype or a product made by the cell population of the second phenotype.

Embodiment 138. The product of embodiment 137, wherein the product includes the subset population of cells of the second phenotype product, and wherein is appropriate for use in clinical therapy.

Embodiment 139. The product of embodiment 137, wherein the product includes a biochemical product that is produced by the subset population of cells of the second phenotype, wherein the biochemical product is appropriate for use in clinical therapy.

In some embodiments, the invention provides a method of culturing a cell for enhanced cytotoxicity comprising culturing the cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure, wherein the cell is a peripheral blood mononuclear cell (PBMC), a pan T-cell, a regulatory T-cell (Treg), or a natural killer (NK) cell. In some embodiments, the oxygen is about 15%. In some embodiments, the pressure condition is at least about 1 PSI above atmospheric pressure. In some embodiments, expression of the cytokine is increased as compared to expression of the cytokine at the culturing condition of about 18% oxygen and 0 PSI above atmospheric pressure. In some embodiments, expression of the cytokine is decreased as compared to expression of the cytokine at the culturing condition of about 18% oxygen and 0 PSI above atmospheric pressure. In some embodiments, the cell is the PBMC. In some embodiments, the cytokine is IL-10, and the expression of IL-10 is decreased. In some embodiments, the cytokine is TNF-α, and the expression of TNF-α is increased. In some embodiments, the cytokine is IL-6, and the expression of IL-6 is decreased. In some embodiments, the cytokine is IFN-γ, and the expression of IFN-γ is increased. In some embodiments, the cytokine is TGF-β1, and the expression of TGF-β1 is increased. In some embodiments, the cytotoxicity of the PBMC is increased by at least about 20% as compared to a cytotoxicity of the PMBC at the control culturing condition of 18% oxygen and 0 PSI above atmospheric pressure. In some embodiments, the cell is the pan T-cell. In some embodiments, the cytokine is IL-6, and the expression of IL-6 is increased. In some embodiments, the cytokine is IFN-γ, and the expression of IFN-γ is increased. In some embodiments, the cytotoxicity of the pan T-cell is increased by at least about 20% as compared to a cytotoxicity of the pan T-cell at the control culturing condition of 18% oxygen and 0 PSI above atmospheric pressure. In some embodiments, expression of a cytotoxicity gene is altered as compared to expression of the cytotoxicity gene at the control culturing condition of 18% oxygen and 0 PSI above atmospheric pressure. In some embodiments, expression of the cytotoxicity gene is increased as compared to expression of the cytotoxicity gene at the culturing condition of about 18% oxygen and 0 PSI above atmospheric pressure. In some embodiments, the cytotoxicity gene is GZMB. In some embodiments, the cytotoxicity gene is perforin. In some embodiments, expression of a checkpoint gene is altered as compared to expression of the checkpoint gene at the control culturing condition of 18% oxygen and 0 PSI above atmospheric pressure. In some embodiments, expression of the checkpoint gene is increased as compared to expression of the checkpoint gene at the culturing condition of about 18% oxygen and 0 PSI above atmospheric pressure. In some embodiments, the checkpoint gene is PD1.

In some embodiments, the invention provides a method of treating a tumor in a subject in need thereof, the method comprising culturing a cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure and after the culturing, administering the cell to the subject wherein the cell is a peripheral blood mononuclear cell (PBMC), a pan T-cell, a regulatory T-cell (Treg), or a natural killer (NK) cell. In some embodiments, the oxygen is about 15%. In some embodiments, the pressure condition is at least about 1 PSI above atmospheric pressure. In some embodiments, expression of the cytokine is increased as compared to expression of the cytokine at the culturing condition of about 18% oxygen and 0 PSI above atmospheric pressure. In some embodiments, expression of the cytokine is decreased as compared to expression of the cytokine at the culturing condition of about 18% oxygen and 0 PSI above atmospheric pressure. In some embodiments, the cell is the PBMC. In some embodiments, cytokine is IL-10, and the expression of IL-10 is decreased. In some embodiments, the cytokine is TNF-α, and the expression of TNF-α is increased. In some embodiments, the cytokine is IL-6, and the expression of IL-6 is decreased. In some embodiments, the cytokine is IFN-γ, and the expression of IFN-γ is increased. In some embodiments, the cytokine is TGF-β1, and the expression of TGF-β1 is increased. In some embodiments, the cell is the pan T-cell. In some embodiments, the cytokine is IL-6, and the expression of IL-6 is increased. In some embodiments, the cytokine is IFN-γ, and the expression of IFN-γ is increased. In some embodiments, cytotoxicity of the cell is increased by at least about 20% as compared to a cytotoxicity of the cell at the control culturing condition of 18% oxygen and 0 PSI above atmospheric pressure. In some embodiments, expression of a cytotoxicity gene is altered as compared to expression of the cytotoxicity gene at the control culturing condition of 18% oxygen and 0 PSI above atmospheric pressure. In some embodiments, expression of the cytotoxicity gene is increased as compared to expression of the cytotoxicity gene at the culturing condition of about 18% oxygen and 0 PSI above atmospheric pressure. In some embodiments, wherein the cytotoxicity gene is GZMB. In some embodiments, the cytotoxicity gene is perforin. In some embodiments, expression of a checkpoint gene is altered as compared to expression of the checkpoint gene at the control culturing condition of 18% oxygen and 0 PSI above atmospheric pressure. In some embodiments, expression of the checkpoint gene is increased as compared to expression of the checkpoint gene at the culturing condition of about 18% oxygen and 0 PSI above atmospheric pressure. In some embodiments, the checkpoint gene is PD1. In some embodiments, the cell is co-administered to the subject with an anti-cancer agent. In some embodiments, the cell is the PBMC. In some embodiments, the anti-cancer agent is a PD1 inhibitor. In some embodiments, the anti-cancer agent is pembrolizumab. In some embodiments, the anti-cancer agent is nivolumab. In some embodiment, the tumor comprises prostate cancer cells.

In some embodiments, the invention provides a method for determining efficacy of an anti-cancer agent, the method comprising: (a) culturing a cell that is selected from the group consisting of peripheral blood mononuclear cell (PBMC), a pan T-cell, a regulatory T-cell (Treg), or a natural killer (NK) cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure and after the culturing, (b) contacting a tumor cell with the cell and the anti-cancer agent; and (c) measuring cytotoxicity against the tumor cell after at least about five days, thereby determining the efficacy of the anti-cancer agent against the tumor cell. In some embodiment, the oxygen is about 15%. In some embodiments, the pressure condition is at least about 1 PSI above atmospheric pressure. In some embodiments, expression of the cytokine is increased as compared to expression of the cytokine at the culturing condition of about 18% oxygen and 0 PSI above atmospheric pressure. In some embodiments, expression of the cytokine is decreased as compared to expression of the cytokine at the culturing condition of about 18% oxygen and 0 PSI above atmospheric pressure. In some embodiments, the cell is the PBMC. In some embodiments, the cytokine is IL-10, and the expression of IL-10 is decreased. In some embodiments, the cytokine is TNF-α, and the expression of TNF-α is increased. In some embodiments, the cytokine is IL-6, and the expression of IL-6 is decreased. In some embodiments, the cytokine is IFN-γ, and the expression of IFN-γ is increased. In some embodiments, the cytokine is TGF-β1, and the expression of TGF-β1 is increased. In some embodiments, the anti-cancer agent is a PD1 inhibitor. In some embodiments, the anti-cancer agent is pembrolizumab. In some embodiments, the anti-cancer agent is nivolumab. In some embodiments, the tumor cell is a prostate tumor cell.

In some embodiments, the invention provides a method of enriching a cell subpopulation from a source population of pan T-cells, the method comprising culturing the source population under 1% to about 15% oxygen and a pressure condition of no more than about 2 PSI above atmospheric pressure, wherein the cell subpopulation comprises CD8+ cells or CD4+ cells. In some embodiments, the cell subpopulation comprises CD8+ cells. In some embodiments, oxygen is about 15% and the pressure condition is about 2 PSI above atmospheric pressure. In some embodiments, the cell subpopulation comprises CD4+ cells. In some embodiments, the oxygen is about 1% and the pressure condition is about 2 PSI above atmospheric pressure.

In some embodiments, the invention provides a method of treating a tumor in a subject in need thereof, the method comprising culturing a cell under about 1% to about 15% oxygen and a pressure condition of no more than about 2 PSI above atmospheric pressure at least until expression of IL-6 and IFN-γ is increased as compared to expression of the IL-6 and IFN-γ at a culturing condition of about 18% oxygen and 0 PSI above atmospheric pressure and after the culturing, administering the cell to the subject wherein the cell is a pan T-cell.

What is claimed is:

1. A method of culturing a cell for enhanced cytotoxicity comprising culturing the cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure, wherein the cell is a peripheral blood mononuclear cell (PBMC), a pan T-cell, a regulatory T-cell (Treg), or a natural killer (NK) cell.

2. The method of claim 1, wherein the oxygen is about 15%.

3. The method of claim 1, wherein the pressure condition is at least about 1 PSI above atmospheric pressure.

4. The method of claim 1, wherein expression of the cytokine is increased as compared to expression of the cytokine at the culturing condition of about 18% oxygen and 0 PSI above atmospheric pressure.

5. The method of claim 1, wherein expression of the cytokine is decreased as compared to expression of the cytokine at the culturing condition of about 18% oxygen and 0 PSI above atmospheric pressure.

6. The method of claim 1, wherein the cell is the PBMC.

7. The method of claim 6, wherein the cytokine is IL-10, and the expression of IL-10 is decreased.

8. The method of claim 6, wherein the cytokine is TNF-α, and the expression of TNF-α is increased.

9. The method of claim 6, wherein the cytokine is IL-6, and the expression of IL-6 is decreased.

10. The method of claim 6, wherein the cytokine is IFN-γ, and the expression of IFN-γ is increased.

11. The method of claim 6, wherein the cytokine is TGF-β1, and the expression of TGF-β1 is increased.

12. The method of claim 6, wherein the cytotoxicity of the PBMC is increased by at least about 20% as compared to a cytotoxicity of the PMBC at the control culturing condition of 18% oxygen and 0 PSI above atmospheric pressure.

13. The method of claim 1, wherein the cell is the pan T-cell.

14. The method of claim 13, wherein the cytokine is IL-6, and the expression of IL-6 is increased.

15. The method of claim 13, wherein the cytokine is IFN-γ, and the expression of IFN-γ is increased.

16. The method of claim 13, wherein the cytotoxicity of the pan T-cell is increased by at least about 20% as compared to a cytotoxicity of the pan T-cell at the control culturing condition of 18% oxygen and 0 PSI above atmospheric pressure.

17. The method of claim 13, wherein expression of a cytotoxicity gene is altered as compared to expression of the cytotoxicity gene at the control culturing condition of 18% oxygen and 0 PSI above atmospheric pressure.

18. The method of claim 17, wherein expression of the cytotoxicity gene is increased as compared to expression of the cytotoxicity gene at the culturing condition of about 18% oxygen and 0 PSI above atmospheric pressure.

19. The method of claim 18, wherein the cytotoxicity gene is GZMB.

20. The method of claim 18, wherein the cytotoxicity gene is perforin.

21. The method of claim 13, wherein expression of a checkpoint gene is altered as compared to expression of the checkpoint gene at the control culturing condition of 18% oxygen and 0 PSI above atmospheric pressure.

22. The method of claim 21, wherein expression of the checkpoint gene is increased as compared to expression of the checkpoint gene at the culturing condition of about 18% oxygen and 0 PSI above atmospheric pressure.

23. The method of claim 22, wherein the checkpoint gene is PD1.

24. A method of treating a tumor in a subject in need thereof, the method comprising culturing a cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure and after the culturing, administering the cell to the subject wherein the cell is a peripheral blood mononuclear cell (PBMC), a pan T-cell, a regulatory T-cell (Treg), or a natural killer (NK) cell.

25. The method of claim 24, wherein the oxygen is about 15%.

26. The method of claim 24, wherein the pressure condition is at least about 1 PSI above atmospheric pressure.

27. The method of claim 24, wherein expression of the cytokine is increased as compared to expression of the cytokine at the culturing condition of about 18% oxygen and 0 PSI above atmospheric pressure.

28. The method of claim 24, wherein expression of the cytokine is decreased as compared to expression of the cytokine at the culturing condition of about 18% oxygen and 0 PSI above atmospheric pressure.

29. The method of claim 24, wherein the cell is the PBMC.

30. The method of claim 29, wherein the cytokine is IL-10, and the expression of IL-10 is decreased.

31. The method of claim 29, wherein the cytokine is TNF-α, and the expression of TNF-α is increased.

32. The method of claim 29, wherein the cytokine is IL-6, and the expression of IL-6 is decreased.

33. The method of claim 29, wherein the cytokine is IFN-γ, and the expression of IFN-γ is increased.

34. The method of claim 29, wherein the cytokine is TGF-β1, and the expression of TGF-β1 is increased.

35. The method of claim 24, wherein the cell is the pan T-cell.

36. The method of claim 35, wherein the cytokine is IL-6, and the expression of IL-6 is increased.

37. The method of claim 35, wherein the cytokine is IFN-γ, and the expression of IFN-γ is increased.

38. The method of claim 24, wherein cytotoxicity of the cell is increased by at least about 20% as compared to a cytotoxicity of the cell at the control culturing condition of 18% oxygen and 0 PSI above atmospheric pressure.

39. The method of claim 35, wherein expression of a cytotoxicity gene is altered as compared to expression of the cytotoxicity gene at the control culturing condition of 18% oxygen and 0 PSI above atmospheric pressure.

40. The method of claim 39, wherein expression of the cytotoxicity gene is increased as compared to expression of the cytotoxicity gene at the culturing condition of about 18% oxygen and 0 PSI above atmospheric pressure.

41. The method of claim 39, wherein the cytotoxicity gene is GZMB.

42. The method of claim 39, wherein the cytotoxicity gene is perforin.

43. The method of claim 35, wherein expression of a checkpoint gene is altered as compared to expression of the checkpoint gene at the control culturing condition of 18% oxygen and 0 PSI above atmospheric pressure.

44. The method of claim 43, wherein expression of the checkpoint gene is increased as compared to expression of the checkpoint gene at the culturing condition of about 18% oxygen and 0 PSI above atmospheric pressure.

45. The method of claim 44, wherein the checkpoint gene is PD1.

46. The method of claim 24, wherein the cell is co-administered to the subject with an anti-cancer agent.

47. The method of claim 46, wherein the cell is the PBMC.

48. The method of claim 47, wherein the anti-cancer agent is a PD1 inhibitor.

49. The method of claim 47, wherein the anti-cancer agent is pembrolizumab.

50. The method of claim 47, wherein the anti-cancer agent is nivolumab.

51. The method of claim 47, wherein the tumor comprises prostate cancer cells.

52. A method for determining efficacy of an anti-cancer agent, the method comprising:
(a) culturing a cell that is selected from the group consisting of peripheral blood mononuclear cell (PBMC), a pan T-cell, a regulatory T-cell (Treg), or a natural killer (NK) cell under about 1% to about 15% oxygen and a pressure condition of no more than about 2 PSI above atmospheric pressure at least until expression of a cytokine is altered as compared to expression of the cytokine at a culturing condition of about 18% oxygen and 0 PSI above atmospheric pressure and after the culturing,
(b) contacting a tumor cell with the cell and the anti-cancer agent; and
(c) measuring cytotoxicity against the tumor cell after at least about five days, thereby determining the efficacy of the anti-cancer agent against the tumor cell.

53. The method of claim 52, wherein the oxygen is about 15%.

54. The method of claim 52, wherein the pressure condition is at least about 1 PSI above atmospheric pressure.

55. The method of claim 52, wherein expression of the cytokine is increased as compared to expression of the cytokine at the culturing condition of about 18% oxygen and 0 PSI above atmospheric pressure.

56. The method of claim 52, wherein expression of the cytokine is decreased as compared to expression of the cytokine at the culturing condition of about 18% oxygen and 0 PSI above atmospheric pressure.

57. The method of claim 52, wherein the cell is the PBMC.

58. The method of claim 57, wherein the cytokine is IL-10, and the expression of IL-10 is decreased.

59. The method of claim 57, wherein the cytokine is TNF-α, and the expression of TNF-α is increased.

60. The method of claim 57, wherein the cytokine is IL-6, and the expression of IL-6 is decreased.

61. The method of claim 57, wherein the cytokine is IFN-γ, and the expression of IFN-γ is increased.

62. The method of claim 57, wherein the cytokine is TGF-β1, and the expression of TGF-β1 is increased.

63. The method of claim 52, wherein the anti-cancer agent is a PD1 inhibitor.

64. The method of claim 52, wherein the anti-cancer agent is pembrolizumab.

65. The method of claim 52, wherein the anti-cancer agent is nivolumab.

66. The method of claim 52, wherein the tumor cell is a prostate tumor cell.

67. A method of enriching a cell subpopulation from a source population of pan T-cells, the method comprising culturing the source population under 1% to about 15% oxygen and a pressure condition of no more than about 2 PSI above atmospheric pressure, wherein the cell subpopulation comprises CD8+ cells or CD4+ cells.

68. The method of claim 67, wherein the cell subpopulation comprises CD8+ cells.

69. The method of claim 68, wherein the oxygen is about 15% and the pressure condition is about 2 PSI above atmospheric pressure.

70. The method of claim 67, wherein the cell subpopulation comprises CD4+ cells.

71. The method of claim 70, wherein the oxygen is about 1% and the pressure condition is about 2 PSI above atmospheric pressure.

72. A method of treating a tumor in a subject in need thereof, the method comprising culturing a cell under about 1% to about 15% oxygen and a pressure condition of no more than about 2 PSI above atmospheric pressure at least until expression of IL-6 and IFN-γ is increased as compared to expression of the IL-6 and IFN-γ at a culturing condition of about 18% oxygen and 0 PSI above atmospheric pressure and after the culturing, administering the cell to the subject wherein the cell is a pan T-cell.

73. A method of modulating a phenotype of at least a subset of a source population of cells, the method comprising: culturing the source cell population in a cell culture incubator that is configured to be able to regulate at least two variable atmospheric condition parameters within the incubator independently of a respective ambient atmospheric condition, wherein two of the variable atmospheric parameters are an oxygen level and a total atmospheric pressure level; regulating at least one of the oxygen level and the total atmospheric pressure level within the incubator such that at least one of the oxygen level or the total atmospheric pressure level differs from the respective ambient level; and as a consequence of the regulating of the variable atmospheric condition parameters, driving expression of a phenotypic parameter of the source population, over an incubation period, from a first phenotype toward a second phenotype, wherein the first phenotype of the subset cell population is that which would be expressed under an atmospheric condition in which the variable atmospheric condition parameters within the incubator were substantially the same as ambient atmospheric conditions, and wherein the second phenotype of the subset cell population is expressed as a consequence of exposure to the variable atmospheric conditions, as regulated by the incubator.
